(19) United States
(12) Patent Application Publication
(10) Pub. No.: US 2026/0165844 A1
(43) Pub. Date: Jun. 18, 2026

(54) GLENOID IMPLANTS, SYSTEMS AND METHODS OF USING THE SAME

(71) Applicants: Patrick Joel DENARD, Medford, OR (US); Alexandre LÄDERMANN, Soral (CH)

(72) Inventors: Patrick Joel DENARD, Medford, OR (US); Alexandre LÄDERMANN, Soral (CH)

(21) Appl. No.: 19/420,191

(22) Filed: Dec. 15, 2025

Related U.S. Application Data
(60) Provisional application No. 63/733,864, filed on Dec. 13, 2024.

(51) Int. Cl. A61F 2/40; A61F 2/30; A61F 2/46

(52) U.S. Cl. CPC A61F 2/4014; A61F 2002/30113; A61F 2002/30242; A61F 2002/30405; A61F 2002/3055; A61F 2002/30565; A61F 2002/30649; A61F 2002/4022; A61F 2/46

(57) ABSTRACT

Implants, devices, systems and methods for replacing an articulation surface in a joint. An implant including a head with an articulating surface and a base surface. An implant including a base member. An implant including an extension/distraction mechanism dynamically connecting the head of the implant to the base member of the implant. A method of inserting an implant into a joint including inserting a base member of the implant into a prepared glenohumeral joint, placing a head of the implant into a glenoid cavity, positioning an extension/distraction mechanism of the implant to dynamically adjust a total length of the implant, and securing the total length of the implant.

### CROSS REFERENCE TO RELATED APPLICATION

This application claims priority benefit under 35 U.S.C. § 119(e) to U.S. Provisional Application No. 63/733,864 filed on Dec. 13, 2024 and entitled Glenoid Implants, Systems and Methods of Using the Same, which is incorporated herein by reference in its entirety.

### FIELD OF THE INVENTION

The present invention relates to general surgery, orthopedic implants used for replacing an articulating surface in a joint, such as shoulder prostheses. More specifically, but not exclusively, the present invention relates to glenoid implants or dynamic glenoid lateralizing and distalizing components, and systems for shoulder replacements and revisions, as well as methods for using the same.

### BACKGROUND OF THE INVENTION

Shoulder replacement is a surgical procedure in which all or part of the glenohumeral joint is replaced by a prosthetic implant. Such joint replacement surgery generally is conducted to relieve arthritis pain or fix severe physical joint damage.

Shoulder replacement surgery is an option for treatment of severe arthritis of the shoulder joint. Arthritis is a condition that affects the cartilage of the joints. As the cartilage lining wears away, the protective lining between the bones is lost. When this happens, painful bone-on-bone arthritis develops. Severe shoulder arthritis is quite painful, and it can cause restriction of motion. While this may be tolerated with some medications and lifestyle adjustments, there may come a time when surgical treatment is necessary.

Common features of one type of a shoulder prosthesis are a Grammont reverse shoulder arthroplasty. A Grammont reverse shoulder arthroplasty typically requires a medialization of the glenohumeral center of rotation, the use of a large ball on the glenoid component, and a lowering of the humerus. These steps increase the deltoid lever arm and provide a space for a less restricted range of motion of the proximal humerus and a stable fulcrum essential for active elevation and stability.

However, the Grammont reverse shoulder arthroplasty risks several complications, for example, the medialized glenoid design may cause limited postoperative range of motion or impingement. To address these problems, one proposed design change includes promoting static lateralization or distalization of the glenoid components. However, even these design modifications still have several limitations. For example, placement of a static lateralized glenosphere can be technically difficult; static glenospheres do not allow soft tissue retensioning after the prosthesis has been placed; when dislocation occurs after the operation, components may require removal to adjust the prosthesis'tension; and static glenospheres may also require multiple offsets, which can increase the number of implants required for the manufacturer. Other issues resulting from a static design include neurological lesions, prosthetic instability, and glenoid loosening.

Therefore, an improved shoulder implant design that results in increased range of motion; allows for intra-and post-operation soft tissue retensioning; allows for simpler lateralization and distalization of the implant intra-and post-operation; results in a reduction of total parts; and reduces neurological lesions, prosthetic instability, and glenoid loosening is needed.

### SUMMARY OF THE INVENTION

Aspects of the present disclosure provide glenoid implants and/or dynamic glenoid lateralizing and distalizing components for shoulder arthroplasties. The present invention also provides for systems and methods for shoulder replacements and revisions.

In one aspect, provided herein is an implant including a head with an articulating surface and a base surface and a base member coupled to a head through an extension/distraction mechanism. The extension/distraction mechanism coupled to the base member.

In another aspect, provided herein is an implant including a head, a base member moveably coupled to the head, and a movement system positioned within a portion of the head and engaging a portion of the base member.

In a further aspect, provided herein is a method of inserting an implant into a joint, including a base member of the implant into a prepared glenohumeral joint, placing a head of the implant into a glenoid cavity, positioning an extension/distraction mechanism of the implant to a desired length to adjust a total length of the implant, and securing a length of the implant and a positioning of the head of the implant relative to the base member of the implant.

These, and other objects, features and advantages of this invention will become apparent from the following detailed description of the various aspects of the invention taken in conjunction with the accompanying drawings.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention. It is emphasized that, in accordance with the standard practice in the industry, various features are not drawn to scale. In fact, the dimensions of the various features may be arbitrarily increased or reduced for clarity of discussion. The foregoing and other objects, features and advantages of the invention are apparent from the following detailed description taken in conjunction with the accompanying drawings in which:

FIG. 1 is a first side exploded view of an embodiment of a glenoid implant, in accordance with an aspect of the present disclosure;

FIG. 2 is a first end view of the glenoid implant of FIG. 1, in accordance with an aspect of the present disclosure;

FIG. 3 is a first side view of the assembled glenoid implant of FIG. 1, in accordance with an aspect of the present disclosure;

FIG. 4 is a first perspective view of a spacer used with the glenoid implant of FIG. 1, in accordance with an aspect of the present disclosure;

FIG. 5 is a second perspective view of the spacer of FIG. 4, in accordance with an aspect of the present disclosure;

FIG. 6 is a top view of the spacer of FIG. 4, in accordance with an aspect of the present disclosure;

FIG. 7 is a side view of the spacer of FIG. 4, in accordance with an aspect of the present disclosure;

FIG. 8 is a first end view of the spacer of FIG. 4, in accordance with an aspect of the present disclosure;

FIG. 9 is a second end view of the spacer of FIG. 4, in accordance with an aspect of the present disclosure;

FIG. 10 is a first perspective exploded view of another embodiment of a glenoid implant, in accordance with an aspect of the present disclosure;

FIG. 11 is a second perspective exploded view of the glenoid implant of FIG. 10, in accordance with an aspect of the present disclosure;

FIG. 12 is a side exploded view of the glenoid implant of FIG. 10 with a spacer, in accordance with an aspect of the present disclosure;

FIG. 13 is a first end exploded view of the glenoid implant of FIG. 12, in accordance with an aspect of the present disclosure;

FIG. 14 is a second end exploded view of the glenoid implant of FIG. 12, in accordance with an aspect of the present disclosure;

FIG. 15 is a first perspective view of the assembled glenoid implant of FIG. 12, in accordance with an aspect of the present disclosure;

FIG. 16 is a second perspective view of the glenoid implant of FIG. 15, in accordance with an aspect of the present disclosure;

FIG. 17 is a first side view of the glenoid implant of FIG. 15, in accordance with an aspect of the present disclosure;

FIG. 18 is a second side view of the glenoid implant of FIG. 15, in accordance with an aspect of the present disclosure;

FIG. 19 is a top view of the glenoid implant of FIG. 15, in accordance with an aspect of the present disclosure;

FIG. 20 is a bottom view of the glenoid implant of FIG. 15, in accordance with an aspect of the present disclosure;

FIG. 21 is a first end view of the glenoid implant of FIG. 15, in accordance with the present disclosure;

FIG. 22 is a second end view of the glenoid implant of FIG. 15, in accordance with an aspect of the present disclosure;

FIG. 23 is a first perspective view of a head of the glenoid implant of FIG. 10, in accordance with an aspect of the present disclosure;

FIG. 24 is a second perspective view of the head of FIG. 23, in accordance with an aspect of the present disclosure;

FIG. 25 is a first end view of the head of FIG. 23, in accordance with an aspect of the present disclosure;

FIG. 26 is a second end view of the head of FIG. 23, in accordance with an aspect of the present disclosure;

FIG. 27 is a side view of the head of FIG. 23, in accordance with an aspect of the present disclosure;

FIG. 28 is a first perspective view of a base member of the glenoid implant of FIG. 10, in accordance with an aspect of the present disclosure;

FIG. 29 is a second perspective view of the base member of FIG. 28, in accordance with an aspect of the present disclosure;

FIG. 30 is a first end view of the base member of FIG. 28, in accordance with an aspect of the present disclosure;

FIG. 31 is a second end view of the base member of FIG. 28, in accordance with an aspect of the present disclosure;

FIG. 32 is a side view of the base member of FIG. 28, in accordance with an aspect of the present disclosure;

FIG. 33 is a side exploded view of another embodiment of a glenoid implant, in accordance with an aspect of the present disclosure;

FIG. 34 is a first perspective view of the glenoid implant of FIG. 33, in accordance with an aspect of the present disclosure;

FIG. 35 is a second perspective view of the glenoid implant of FIG. 33, in accordance with an aspect of the present disclosure;

FIG. 36 is a first side view of the glenoid implant of FIG. 33, in accordance with an aspect of the present disclosure;

FIG. 37 is a first end view of the glenoid implant of FIG. 33, in accordance with an aspect of the present disclosure;

FIG. 38 is a second end view of the glenoid implant of FIG. 33, in accordance with an aspect of the present disclosure;

FIG. 39 is a top view of the glenoid implant of FIG. 33, in accordance with an aspect of the present disclosure;

FIG. 40 is a bottom view of the glenoid implant of FIG. 33, in accordance with an aspect of the present disclosure;

FIG. 41 is a top view of a base member of the glenoid implant of FIG. 33, in accordance with an aspect of the present disclosure;

FIG. 42 is a side view of a distraction implement, in accordance with an aspect of the present disclosure;

FIG. 43 is an exploded, first perspective view of another embodiment of a glenoid implant, in accordance with an aspect of the present disclosure;

FIG. 44 is an exploded, second perspective view of the glenoid implant of FIG. 43, in accordance with an aspect of the present disclosure;

FIG. 45 is an exploded, first side view of the glenoid implant of FIG. 43, in accordance with an aspect of the present disclosure;

FIG. 46 is an exploded, second side view of the glenoid implant of FIG. 43, in accordance with an aspect of the present disclosure;

FIG. 47 is an exploded, first side view of the glenoid implant of FIG. 43, in accordance with an aspect of the present disclosure;

FIG. 48 is an exploded, second side view of the glenoid implant of FIG. 43, in accordance with an aspect of the present disclosure;

FIG. 49 is an exploded, top view of the glenoid implant of FIG. 43, in accordance with an aspect of the present disclosure;

FIG. 50 is an exploded, bottom view of the glenoid implant of FIG. 43, in accordance with an aspect of the present disclosure;

FIG. 51 is a first perspective view of an assembled glenoid implant of FIG. 43 in a first position, in accordance with an aspect of the present disclosure;

FIG. 52 is a second perspective view of the glenoid implant of FIG. 51, in accordance with an aspect of the present disclosure;

FIG. 53 is a first side view of the glenoid implant of FIG. 51, in accordance with an aspect of the present disclosure;

FIG. 54 is a first cross-sectional view of the glenoid implant of FIG. 51 taken along line 54-54 in FIG. 60, in accordance with an aspect of the present disclosure;

FIG. 55 is a second side view of the glenoid implant of FIG. 51, in accordance with an aspect of the present disclosure;

FIG. 56 is a second cross-sectional view of the glenoid implant of FIG. 51 taken along line 56-56 in FIG. 60, in accordance with an aspect of the present disclosure;

FIG. 57 is a first end view of the glenoid implant of FIG. 51, in accordance with an aspect of the present disclosure;

FIG. 58 is a second end view of the glenoid implant of FIG. 51, in accordance with an aspect of the present disclosure;

FIG. 59 is a bottom view of the glenoid implant of FIG. 51, in accordance with an aspect of the present disclosure;

FIG. 60 is a top view of the glenoid implant of FIG. 51, in accordance with an aspect of the present disclosure;

FIG. 61 is a top cross-sectional view of the glenoid implant of FIG. 51 taken along line 61-61 in FIG. 57, in accordance with an aspect of the present disclosure;

FIG. 62 is a first perspective view of an assembled glenoid implant of FIG. 43 in a second position, in accordance with an aspect of the present disclosure;

FIG. 63 is a second perspective view of the glenoid implant of FIG. 62, in accordance with an aspect of the present disclosure;

FIG. 64 is a first side view of the glenoid implant of FIG. 62, in accordance with an aspect of the present disclosure;

FIG. 65 is a first cross-sectional view of the glenoid implant of FIG. 62 taken along line 65-65 in FIG. 62, in accordance with an aspect of the present disclosure;

FIG. 66 is a second side view of the glenoid implant of FIG. 62, in accordance with an aspect of the present disclosure;

FIG. 67 is a second cross-sectional view of the glenoid implant of FIG. 62 taken along line 67-67 in FIG. 63, in accordance with an aspect of the present disclosure;

FIG. 68 is a first end view of the glenoid implant of FIG. 62, in accordance with an aspect of the present disclosure; and

FIG. 69 is a second end view of the glenoid implant of FIG. 62, in accordance with an aspect of the present disclosure;

### DETAILED DESCRIPTION FOR CARRYING OUT THE INVENTION

Generally stated, disclosed herein are glenoid implants for shoulder prostheses and systems to aid in dynamically adjusting the glenoid implants. Further, surgical methods for inserting and using the glenoid implants and systems are discussed.

In this detailed description and the following claims, the words proximal, distal, anterior, posterior, medial, lateral, superior and inferior are defined by their standard usage for indicating a particular part of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

As used herein, the word “exemplary” or “illustrative” means “serving as an example, instance, or illustration.” Any implementation described herein as “exemplary” or “illustrative” is not necessarily to be construed as preferred or advantageous over other implementations. Moreover, in the present description, the terms “upper,” “lower,” “left,” “rear,” “right,” “front,” “vertical,” “horizontal,” and derivatives thereof shall relate to the invention as oriented in the first figure of each embodiment.

Similarly, positions or directions may be used herein with reference to anatomical structures or surfaces. For example, as the current implants, devices, systems and methods are described herein with reference to use with the bones of the shoulder, the bones of the shoulder and upper arm may be used to describe the surfaces, positions, directions or orientations of the implants, devices, systems and methods. Further, the implants, devices, systems and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, systems and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, systems and methods, and the aspects, components, features and the like thereof, described herein with respect to the right shoulder may be mirrored so that they likewise function with the left shoulder and vice versa. Further, the implants, devices, systems and methods, and the aspects, components, features and the like thereof, disclosed herein are described with respect to the shoulder for brevity purposes, but it should be understood that the implants, devices, systems and methods may be used with other bones of the body having similar structures, for example the hip.

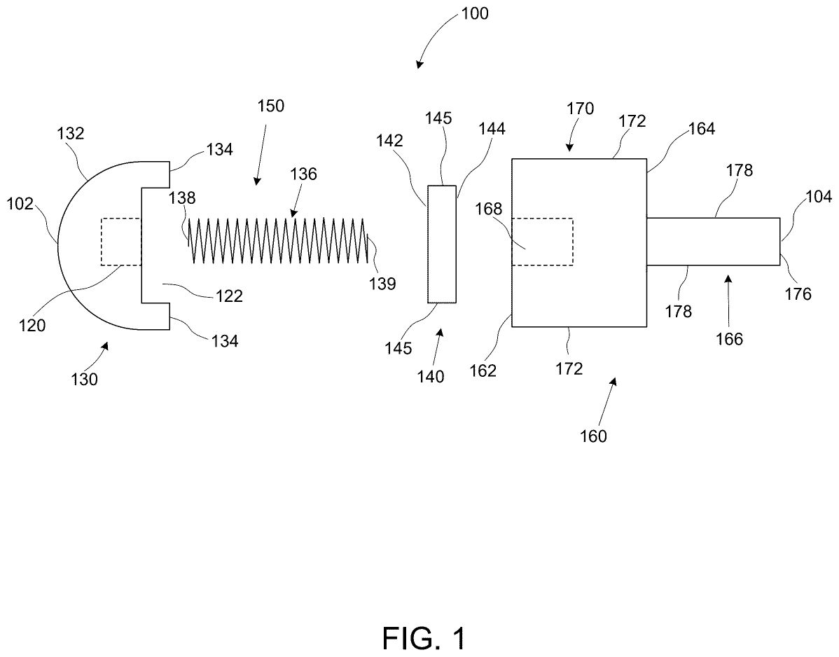

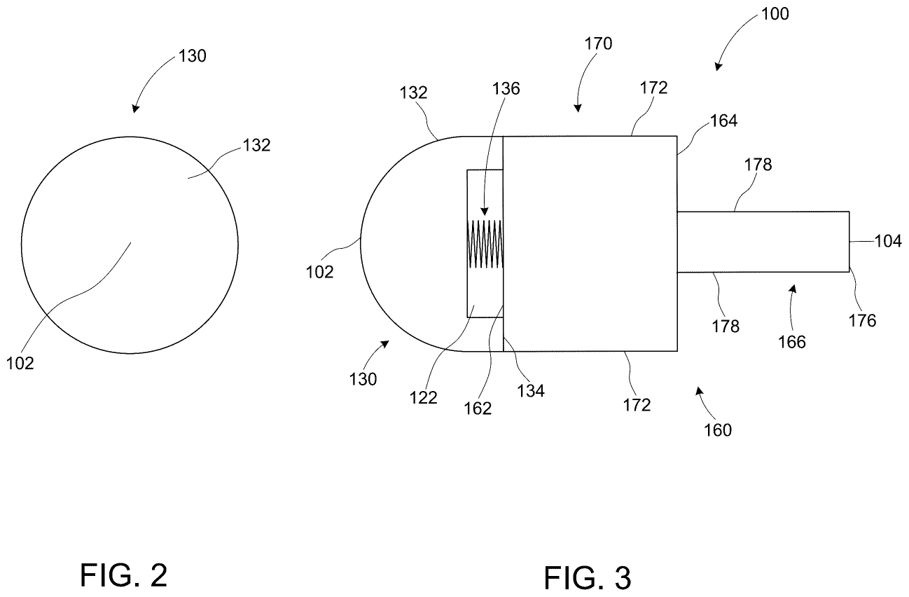

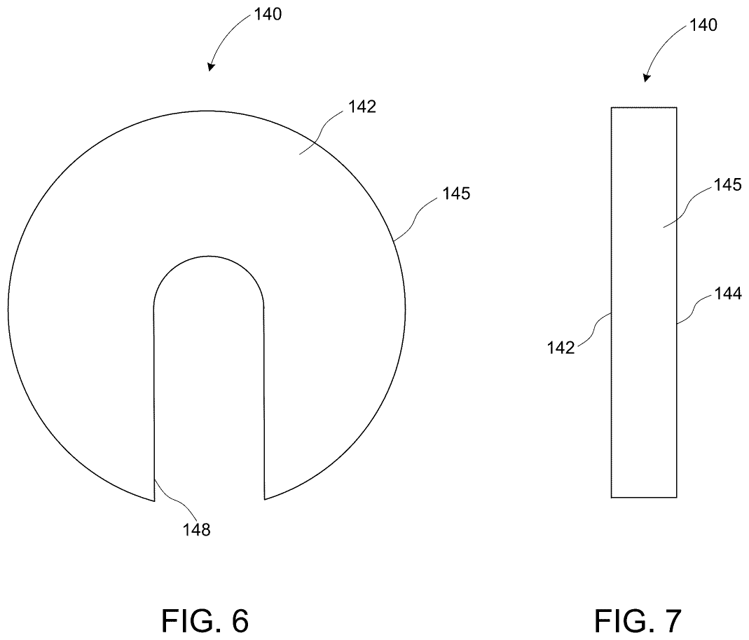

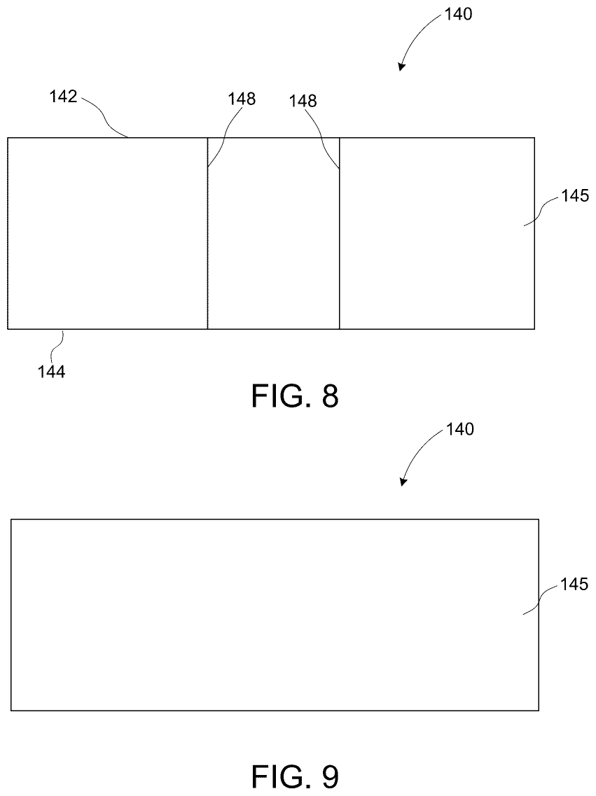

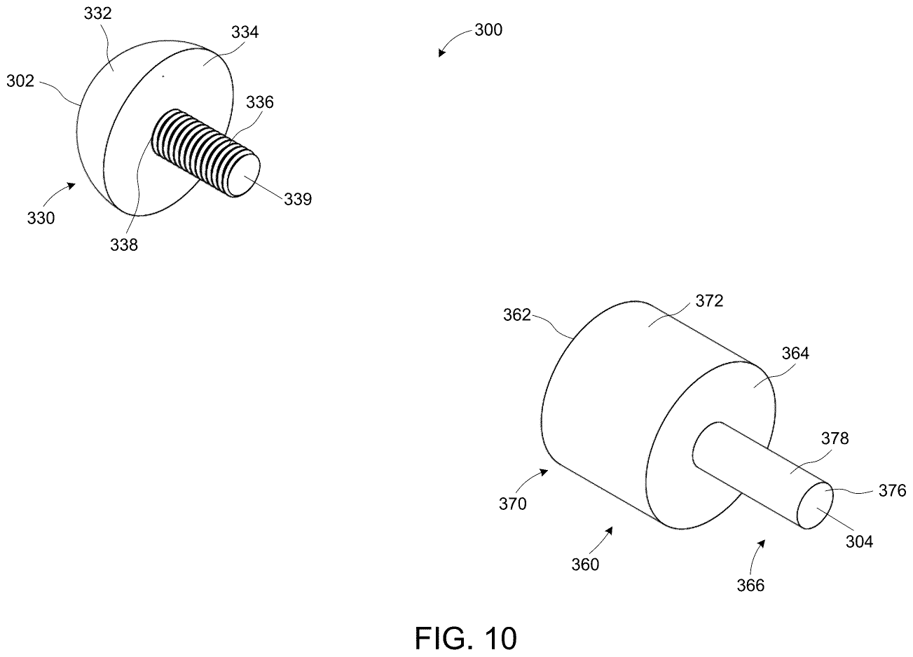

Referring to the drawings, wherein like reference numerals are used to indicate like or analogous components throughout the several views, and with particular reference to FIGS. 1-9, there is illustrated an embodiment of an implant 100 for a glenoid implant system, which may be a traditional or reverse glenoid implant system. The implant 100 may have a head 130, a base member 160, an extension/distraction mechanism 150, and a spacer 140.

The implant 100 may have a first end 102 at an end of the head 130 and a second end 104 at an opposite end of the base member 160.

With continued reference to FIGS. 1-3, the head 130 may have an articulating surface 132 and a base surface 134 opposite and extending between the articulating surface 132 on an interior surface of the head 130. The articulating surface 132 may have, for example, a hemispherical surface. The base surface 134 may include, for example, a first cavity 120 and a second cavity 122 extending into the base surface 134 towards the first end 102 of the implant 100. The first cavity 120 may extend into the head 130 a first distance and the second cavity 122 may extend into the head 130 a second distance. The first distance may be, for example, larger than the second distance. The first cavity 120 and the second cavity 122 may be, for example, cylindrical, where the second cavity 122 may be concentrically aligned with the first cavity 120. The first cavity 120 may have a first diameter and the second cavity 122 may have a second diameter. The second diameter of the second cavity 122 may be larger than the first diameter of the first cavity 120. The larger second diameter of the second cavity 120 may allow for distalization and lateralization of the head 134 relative to the spring 136. The base surface 134 may have, for example, multiple flat surfaces that are recessed at various distances.

The head 130 may be coupled to the extension/distraction mechanism 150 on the base surface 134. The extension/distraction mechanism 150 may include, for example, a spring 136 with a first end 138 and a second end 139. The first end 138 of the spring 136 may be either coupled approximately centrally to the base surface 134 or received within the first cavity 120 of the head 130. In the embodiment where the spring 136 is received within the first cavity 120, the head 130 may further include the second cavity 122.

The base member 160 may include a body 170 and a shaft 166 extending from a second end of the body 170. The body 170 may have a first surface 162 at a first end, a second surface 164 at the second end, and a third surface 172 extending circumferentially around the body between the first surface 162 and the second surface 164. The body 170 may have, for example, an approximately cylindrical shape where the first surface 162 has an approximately circular flat surface and the second surface 164 has an approximately circular flat surface. The first surface 162 may be offset approximately parallel to the second surface 164. The first surface 162 and the second surface 164 may be connected by the third surface 172. The second end 139 of the spring 136 may be coupled to the first surface 162, and the spring 136 may be coupled to the approximate center of first surface 162. Alternatively, instead of the spring 136 coupling to the first surface 162, the body 170 may further include a cavity 168. The cavity 168 may extend from the first surface 162 towards the second surface 164. The cavity 168 may be configured to accept the spring 136, for example, with a diameter that is either the same size or slightly larger than the diameter of the spring 136. In this example, the second end 139 of the spring 136 may be coupled to an end or an interior most surface of the cavity 168. The shaft 166 may have a first surface 176 and a second surface 178. The shaft 166 may be approximately concentrically coupled to the second surface 164 of the body 170 by the second surface 178 which extends approximately normal to the second surface 164 towards the second end 104. The shaft 166 may have a circular first surface 178 which closes off the second surface 178 and may be approximately parallel to the second surface 164 of the body 170. The shaft 166 may be, for example, smooth, threaded along at least a portion, and/or include at least one protrusion along the length.

The head 130 may be separated from the base member 160 by extending the spring 136. The spring 136 may have a sufficiently high spring constant such that extending the spring 136 requires the use of a tool, i.e. a distractor 700. The spring 136 may be capable of holding its distraction when extended.

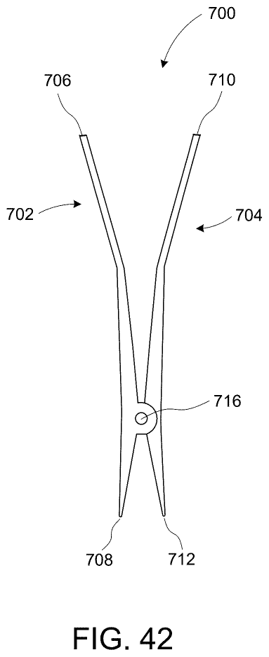

Referring to FIG. 42, the distractor 700 may have a first arm 702 and a second arm 704 pivotably connected with a pin 716 located on the first arm 702. The first arm 702 may have a first end 706 and a second end 708 located opposite the first end 706. The second arm 704 may have a first end 710 and a second end 712 located opposite the first end 710. A distance between the first end 706 of the first arm 702 to the pin 716 and a distance between the first end 710 of the second arm 704 to the pin 716 may be larger or smaller than the distance between the second end 708 of the first arm 702 and a distance between the second end 712 of the second arm 704 to create a lever that either increases or decreases the mechanical force applied from the user to the resulting force applied to the implant 100. In an example of using the distractor 700 to distract the spring 136, a user may position the second end 708 on the base surface 134 of the head 130 and the second end 712 on the first surface 162 of the base member 160. A user may then compress the first arm 702 towards the second arm 704 with their hands or some other tool to extend the distance between the second end 708 of the first arm 702 and the second end 712 of the second arm 704. This extension between the second end 708 and the second end 712 may provide opposing forces on the head 130 and the base member 160, distracting the spring 136.

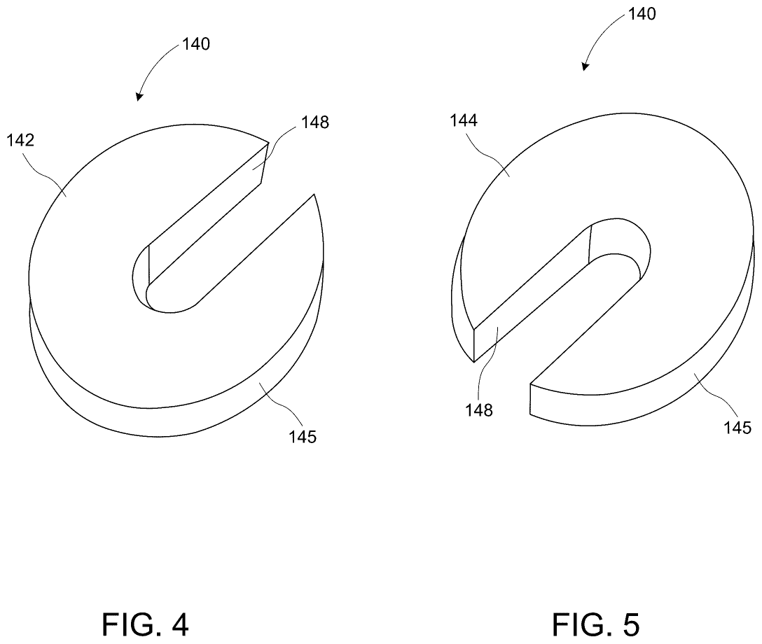

Referring to FIGS. 4-9, the implant 100 may include a spacer 140. The spacer 140 may include a first surface 142 at an end of the spacer 140, a second surface 144 at an opposite end of the spacer 140, and a third surface 145 extending circumferentially around the spacer 140 between the first surface 142 and the second surface 144. The spacer 140 may also include a cavity 146 extending from the first surface 142 to the second surface 144 forming an interior surface 148. The interior surface 148 may extend between the first surface 142 and the second surface 144 along a perimeter of the cavity 146 and from the third surface 145 toward and/or beyond a central position of the spacer 140. The spacer 140 may approximately have a disk shape where the first surface 142 and the second surface 144 have an approximately circular flat shape along the faces of the disk and the third surface 145 connects the outer edges of the first surface 142 and the second surface 144. A distance between the first surface 142 and the second surface 144, (i.e., a length of the third surface 144) may have a plurality of lengths. The cavity 146 may be configured for the spacer 140 to be releasably placed around the spring 136 to extend a distance between the first end 102 and the second end 104 of the implant 100. The cavity 146 may be defined by the interior surface 148, which may start from, for example, a semicircle around or near the approximate center of the first surface 142, that further extends normally from the outer edges of the semicircle towards edges of the first surface 142, further extending through to the second surface 144.

The cavity 146 may have, for example, a width between the interior surfaces 148 that is approximately the same size or slightly larger than the diameter of the spring 136. A diameter of the first surface 142 and the second surface 144 may have an approximately similar diameter to a diameter of the base surface 134 of the head 130 and a diameter of the first surface 162 of the base member 160, such that the spacer 140 can couple to or be positioned between the head 130 and the base member 160.

When the spring 136 has been extended as described above, the cavity 146 of the spacer 140 may be placed around the spring 136. The first surface 142 of the spacer 140 may be aligned with and coupled to the base surface 134 of the head 130 and the second surface 144 of the spacer 140 may be aligned with and coupled to the first surface 162 of the base member 160 to increase a length of the implant 100. Alternatively, multiple spacers 140 may be inserted between the base surface 134 of the head 130 and the first surface 162 of the base member 160 based on the desired distracted length of the implant 100. The spacer 140 may couple to the base surface 134 and the first surface 162 or to another spacer 140 through friction, pins and holes, a clasping mechanism, grooves, screws, metal joining methods, snap-fit, adhesives, glues/epoxies, and/or other types of coupling mechanisms. The spacer 140 may be coupled to similar parts of other embodiments of implants through similar mechanisms.

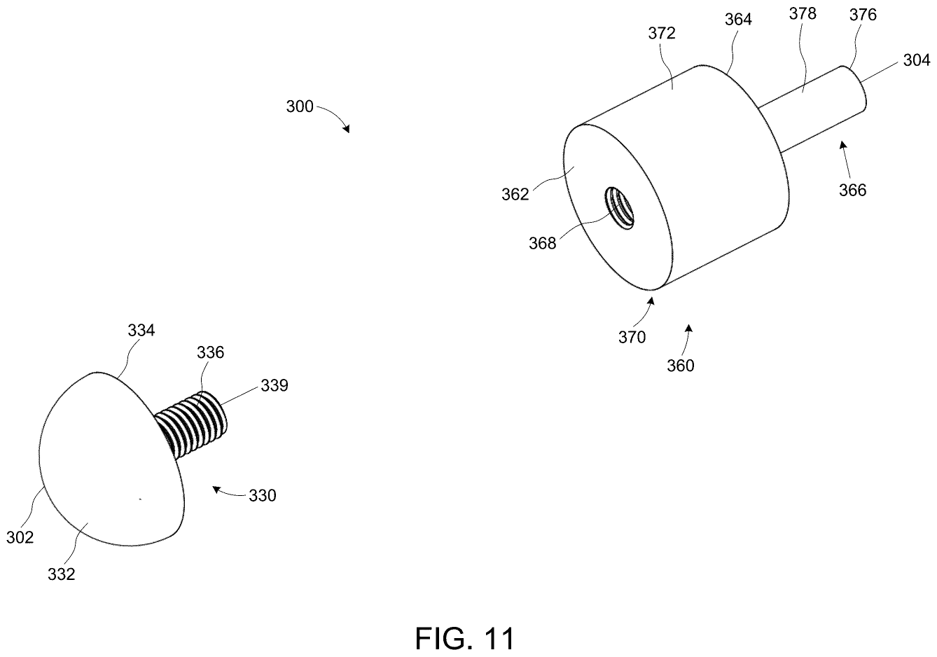

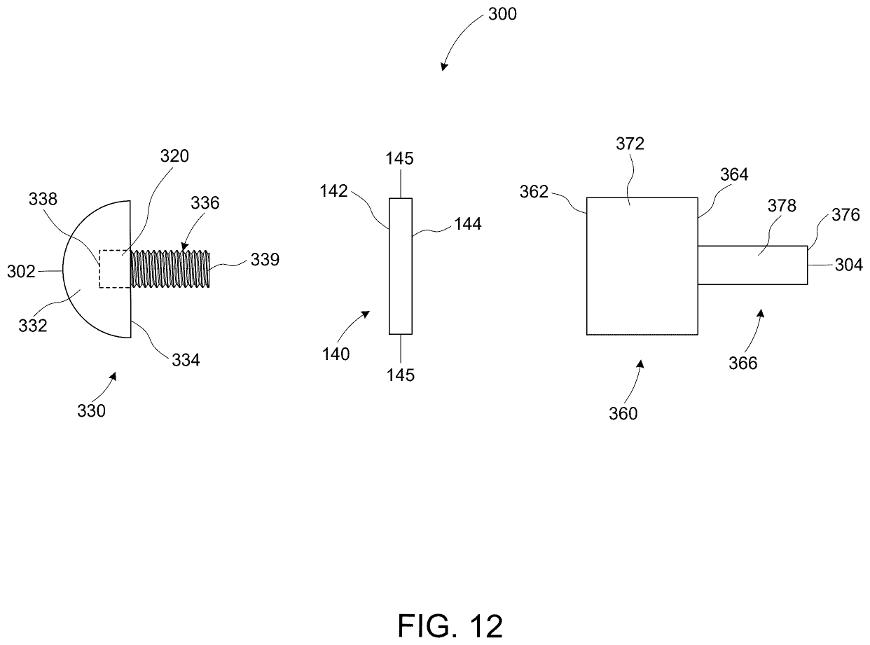

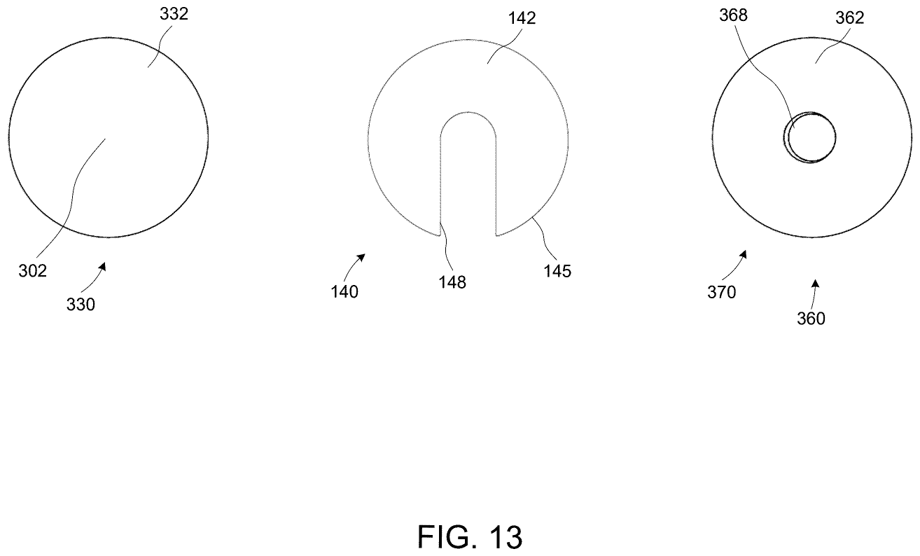

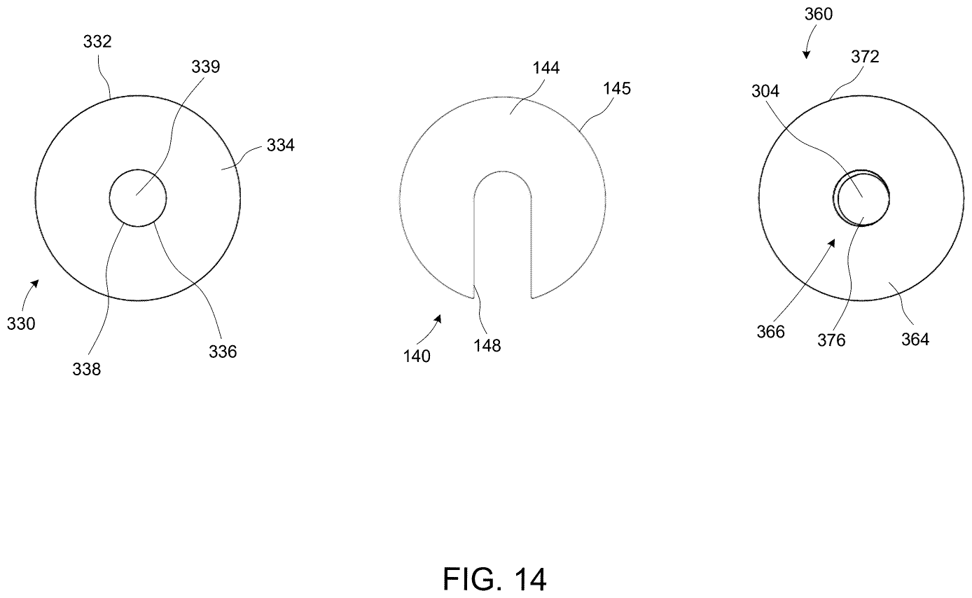

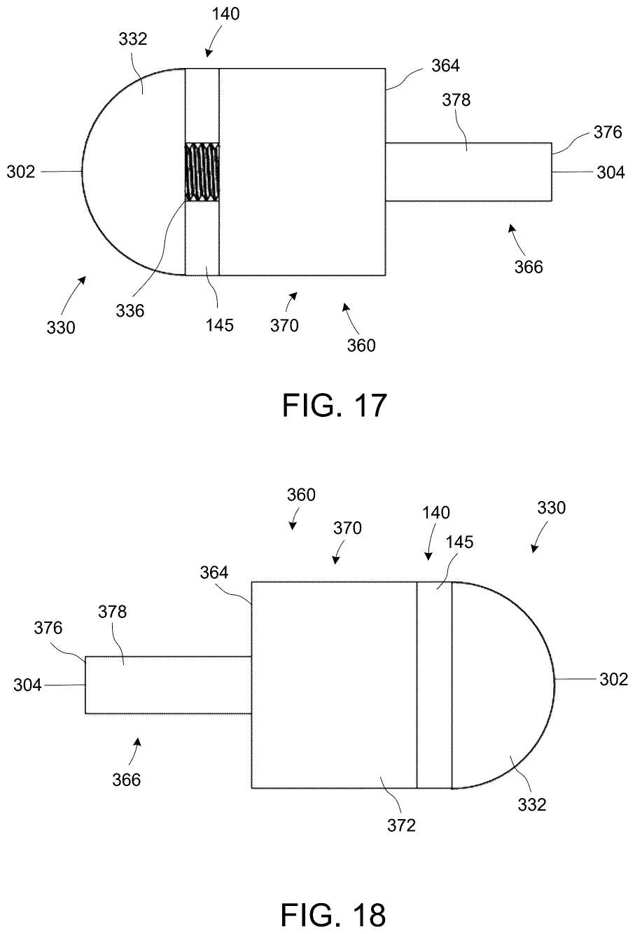

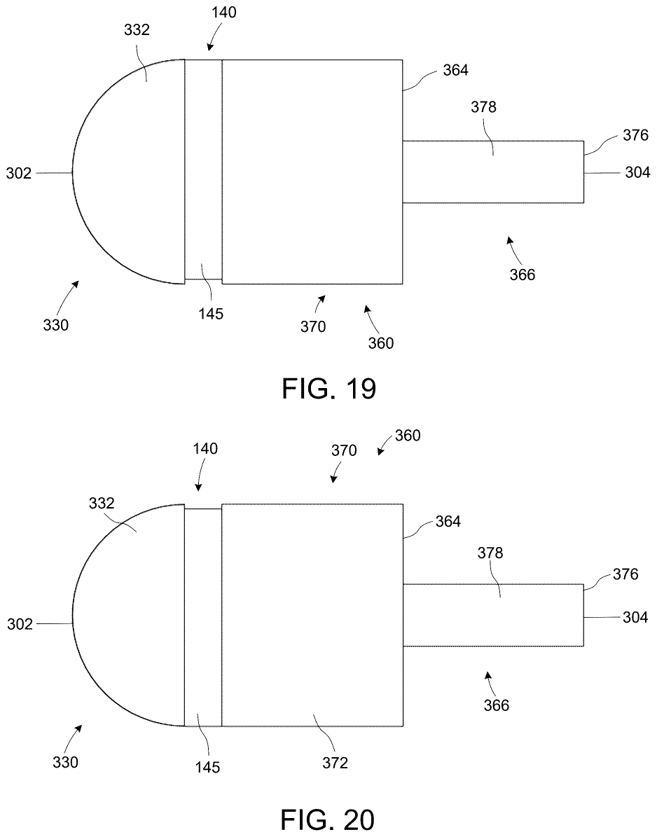

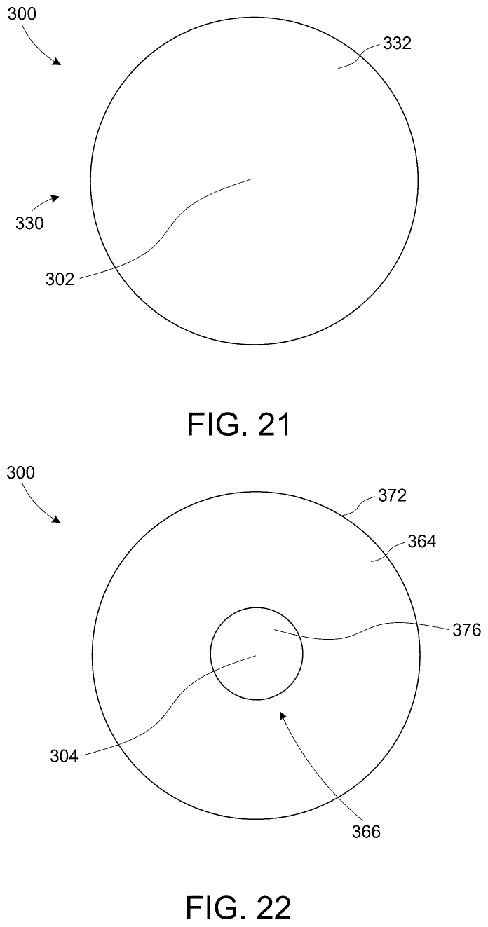

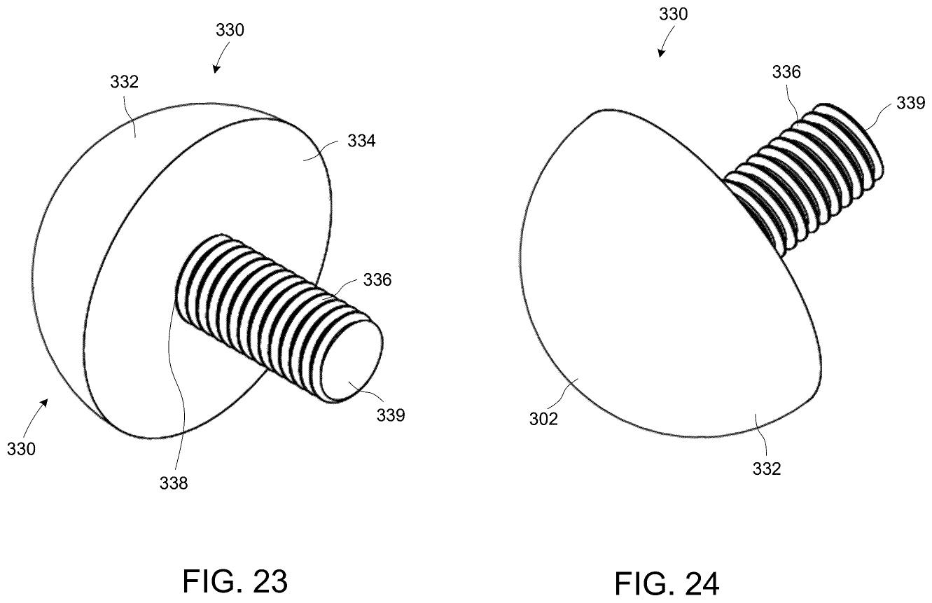

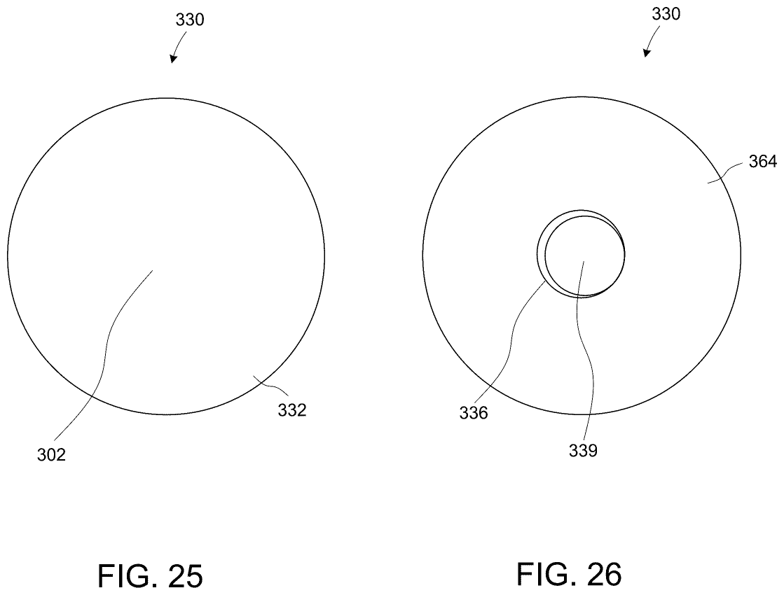

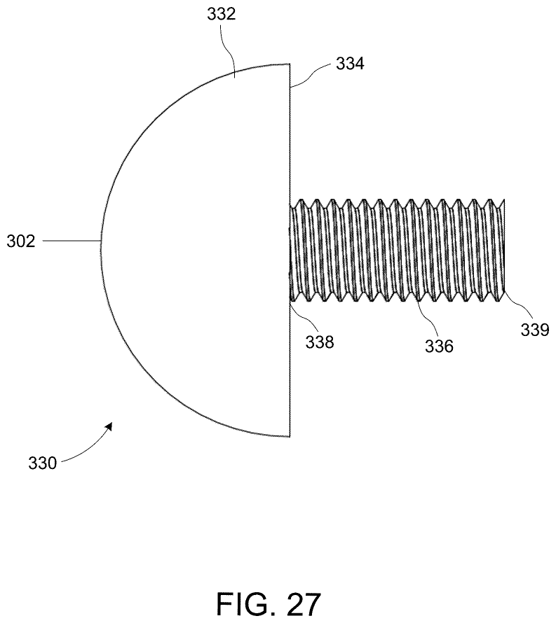

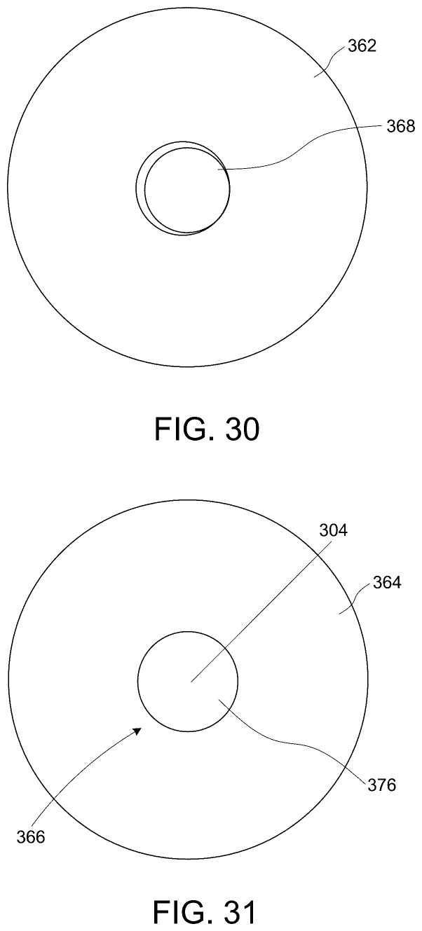

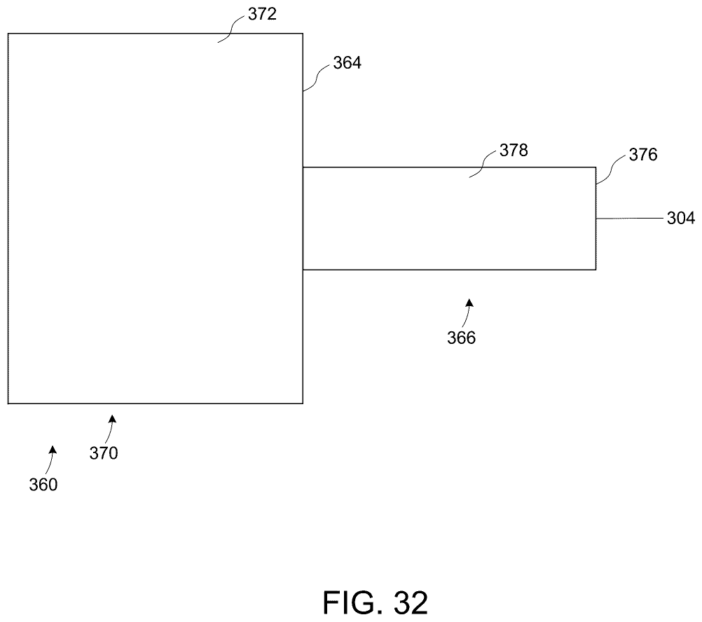

Referring now to FIGS. 10-32, there is illustrated an alternative embodiment of an implant 300 for a glenoid implant system or reverse glenoid implant system. Contrasting the implant 100, the implant 300 may be similar to the implant 100 with corresponding features offset by 200. For example, the first end 102 and the second end 104 of the implant 100 may correspond to a first end 302 and a second end 304 of the implant 300. However, the implant 300 may have an extension/distraction mechanism 350 that operates with a screw 336 instead of the spring 136; therefore, a head 330 may differ in part from the head 130 and a base member 360 may differ in part from the base member 160.

In one illustrative but not limiting example, referring to FIGS. 23-27, the head 330 may further include the screw 336. The screw 336 may be a threaded rod and may be coupled approximately centrally to and extend from the base surface 334 of the head 330 or received and coupled within a cavity 320 of the head 330. The screw 336 may be, for example, threaded along a portion or the entire length. The cavity 320 may either be cylindrical or some other shape to receive a portion of the screw 336 and extend approximately perpendicularly from the first surface 334 of the head 330 towards the first end 302 of the implant 300. The cavity 320 may have a diameter or a cross-sectional area to receive a portion of the screw 336 and may be at least partially threaded to receive a portion of the screw 336. The screw 336 may extend from the base surface 334 or the cavity 320 towards the second end 304 of the implant 300.

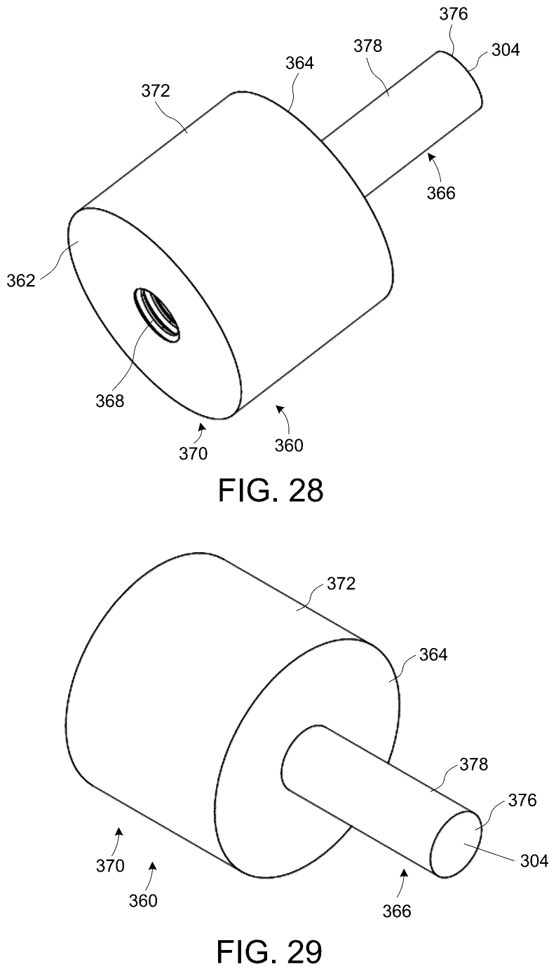

In one illustrative but not limiting example, referring to FIGS. 28-32, the base member 360 may have a threaded cavity 368 that extends from a first surface 362 of a body 370 towards a second surface 364 of the body 360. The threaded cavity 368 may have threads and a diameter or a cross-sectional area to releasably receive screw 336. The cavity 368 may have threads, for example, along the entire length of the cavity 368 or only a portion of the cavity 368.

Referring to FIGS. 10-12, a length of the implant 300 between the first end 302 and the second end 304 may be adjusted by screwing and/or un-screwing the screw 336 into/from the threaded cavity 368 with either a user's hands or a tool.

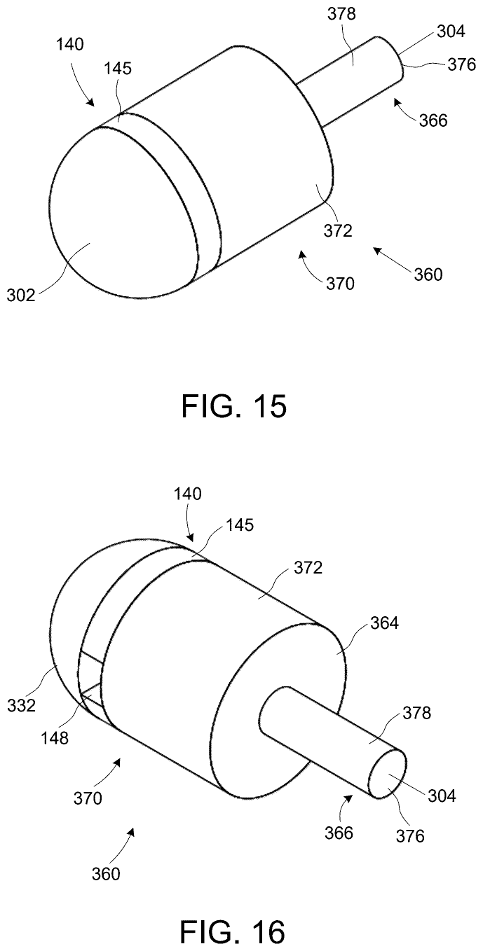

Referring to FIGS. 15-20, the implant 300 may also include the spacer 140, as described in greater detail above with reference to FIGS. 1-9, which will not be described again here again for brevity's sake. When the screw 336 has been extended and thus a distance between the first surface 334 of the head 330 and a first surface 362 of the base member 360 has been extended as described above, the interior surface 148 of the spacer 140 may be placed around the screw 336. The first surface 142 of the spacer 140 may be aligned with and coupled to the first surface 334 of the head 330 and the second surface 144 of the spacer 140 may be aligned with and coupled to the first surface 362 of the base member 360. Alternatively, multiple spacers 140 may be inserted between the first surface 334 of the head 330 and the first surface 362 of the base member 360 based on the distracted length of the implant 300. Once the desired number of spacers 140 is inserted between the head 330 and the base member 360, the head 330 and the base member 360 may be screwed together to secure the spacers 140 in place.

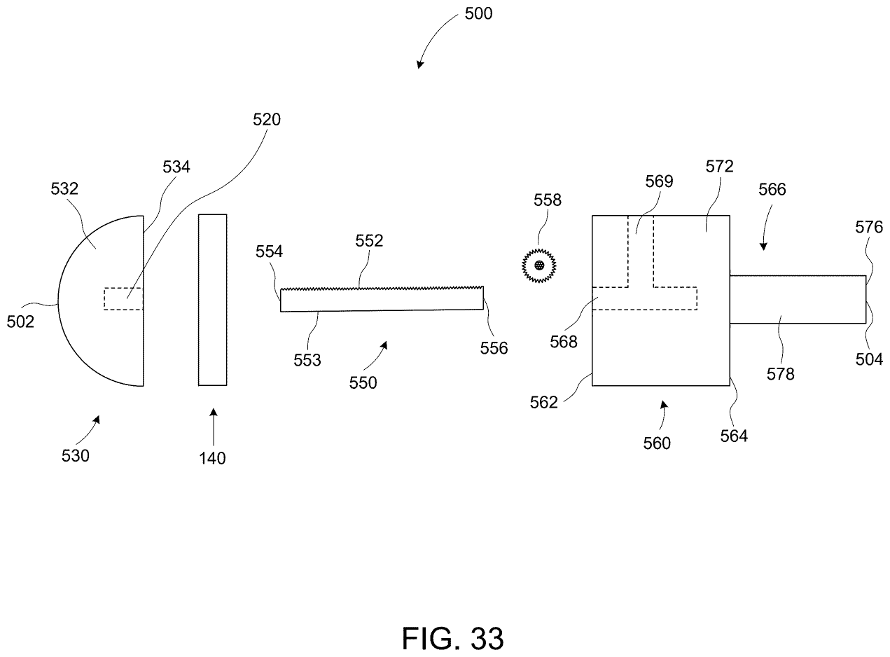

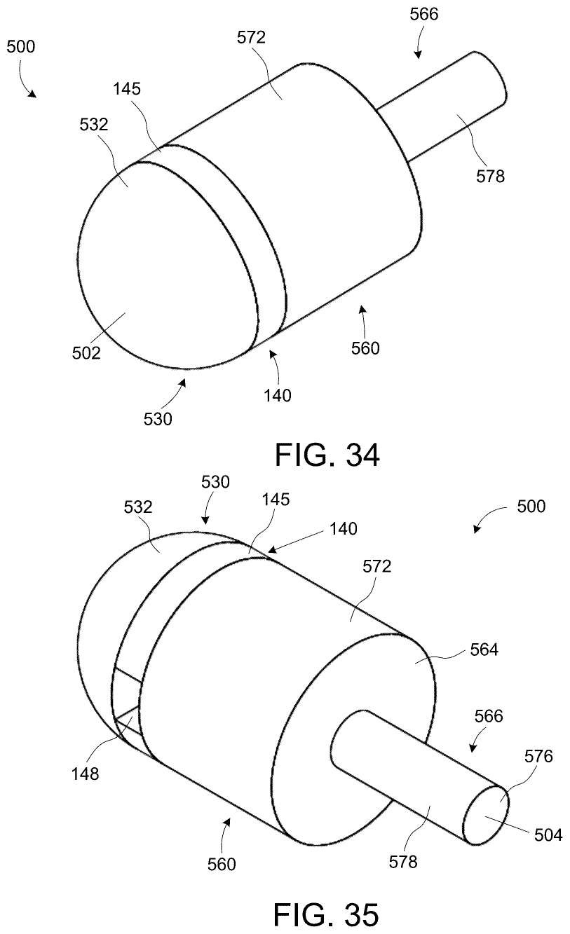

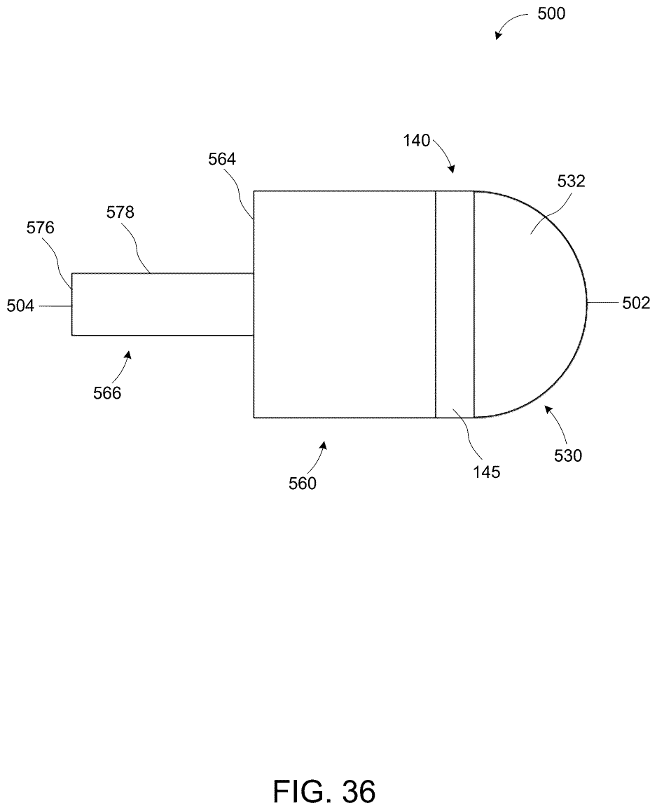

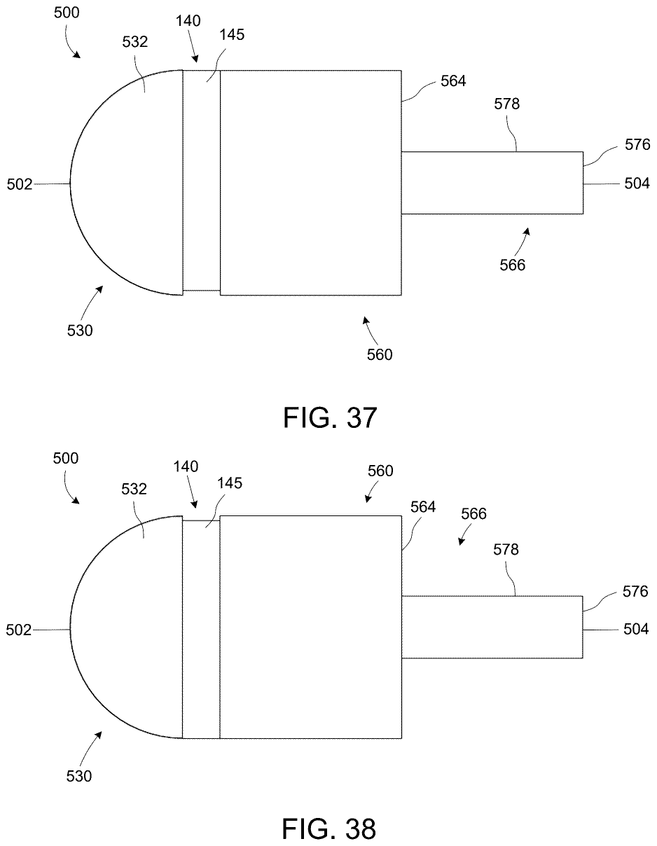

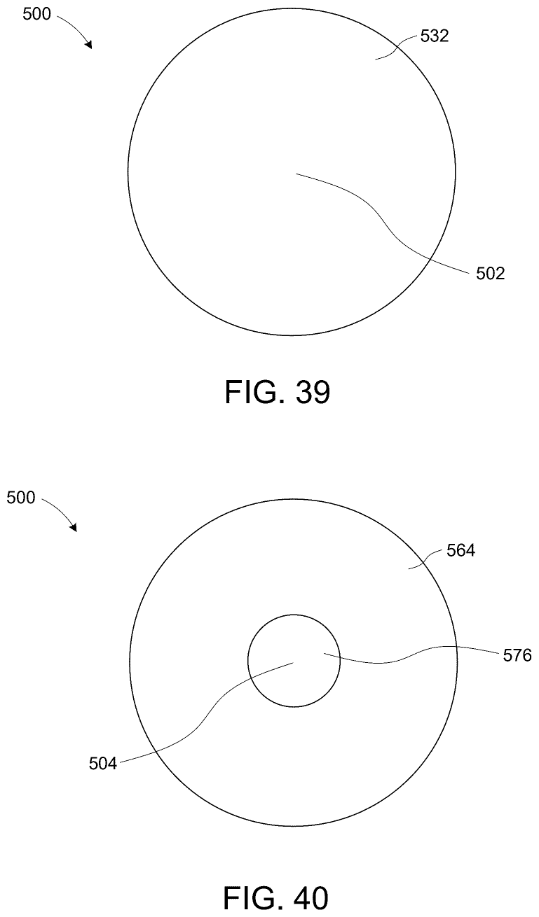

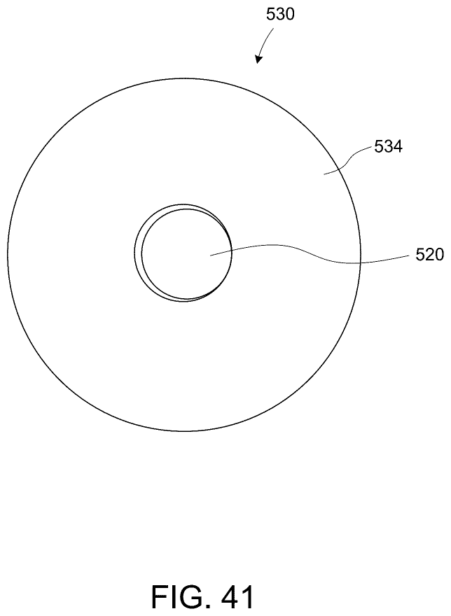

Referring now to FIGS. 33-41, there is illustrated an alternative embodiment of an implant 500 for a glenoid implant system or reverse glenoid implant system. The implant 500 may be similar to the implant 100 with corresponding features offset by 400. For example, the first end 102 and the second end 104 of the implant 100 may correspond to a first end 502 and a second end 504 of the implant 500. However, the implant 500 may have an extension/distraction mechanism 550 that operates with a rack 551 and a gear 558 instead of the spring 136; therefore, a head 530 may differ in part from the head 130 and a base member 560 may differ in part from the base member 160.

Referring to FIG. 33, in one illustrative but not limiting example, the rack 550 may have a first end 554 and an opposing second end 556. In between the first end 554 and the second end 556, the rack 550 may have a teeth side 552 that engages with the gear 558.

The rack 550 may have a second side 553 opposite the teeth side 552. The second side 553 may have, for example, a smooth surface. The first end 554 may be coupled to a base surface 534 of a head 530 or received within or coupled to a cavity 520 of the head 530.

A first cavity 568 of the base member 560 may be configured to releasably receive the second end 556 of the rack 550. The base member 560 may further include a second cavity 569 that may be configured for the gear 558 to be positioned within and for the rack 550 to translate through. The first cavity 568 and the second cavity 569 may be connected such that the rack 550 and the gear 558 may interact within the implant 500. The first cavity 568 may be positioned, for example, generally perpendicular to the second cavity 569.

The coupling of the rack 550 to a first surface 534 of the head 530 along with the connection between the rack 550 and the gear 558 may fix the translation of the rack 550 along a single plane. Furthermore, the head 530 may be separated from the base member 560 by translating the rack 550 with either a user's hands or a tool. For example, a tool may be inserted into the second cavity 569 to engage the gear 558 and move the head 530 relative to the base member 560. The implant 500 may be expanded to extend the length of the implant 500 between the first end 502 and the second end 504 to fit within a patient's anatomy. If the implant 500 is expanded, the space between the head 530 and the base member 560 may be filled with at least one spacer 140. The spacers 140 may have different lengths to fill the space between the head 530 and the base member 560 and/or multiple spacers 140 may be used to fill the space between the head 530 and the base member 560.

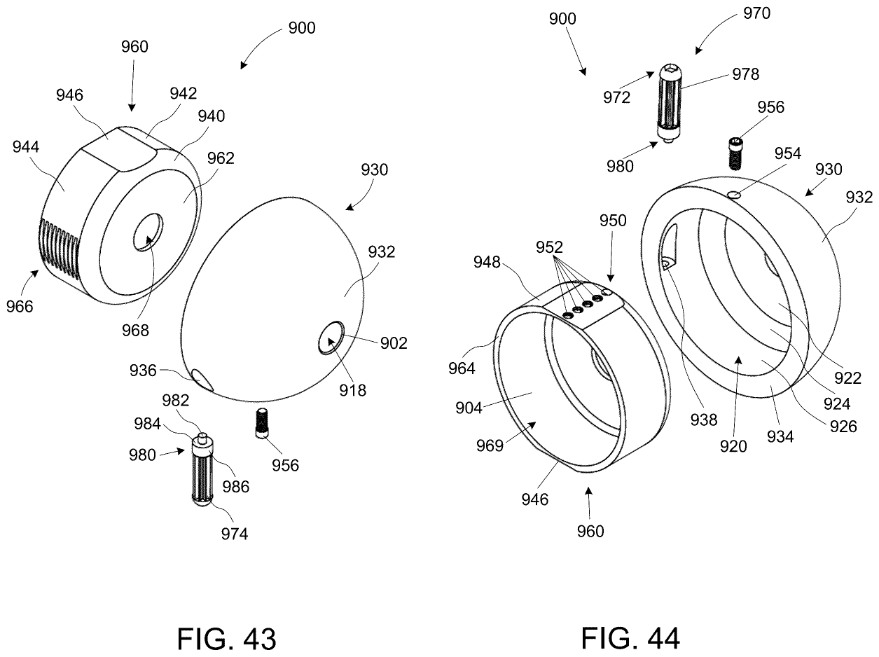

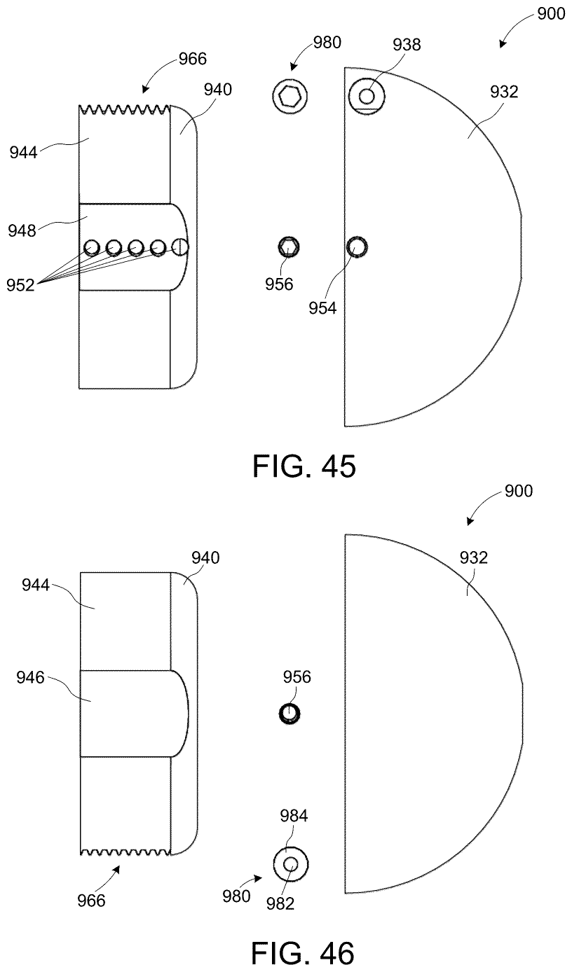

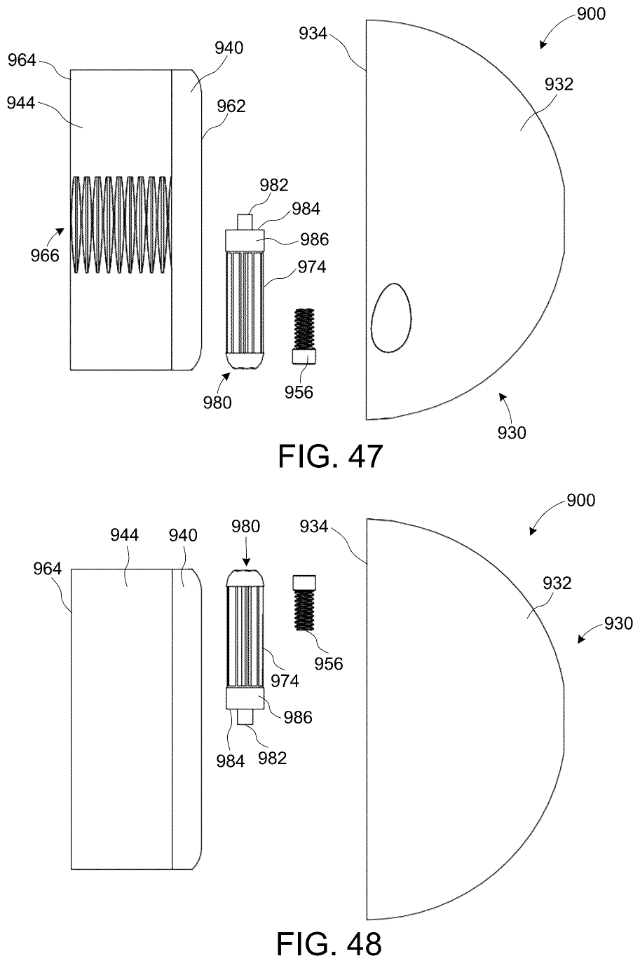

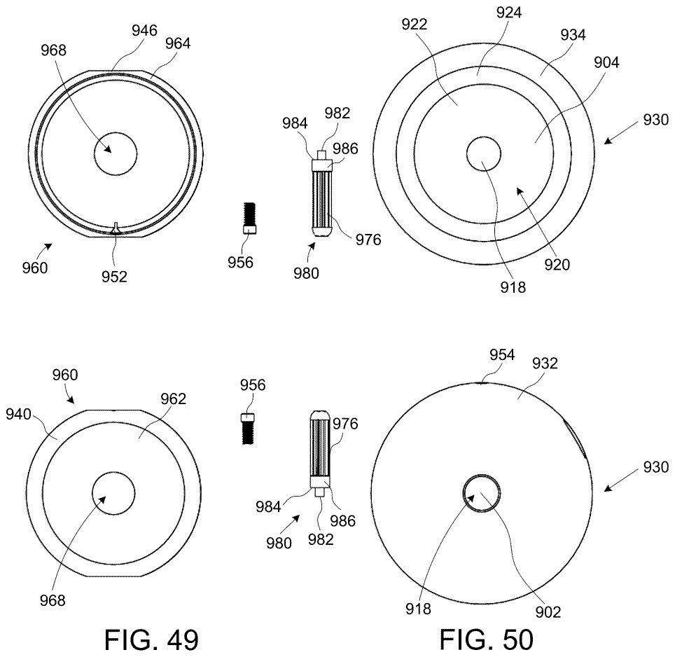

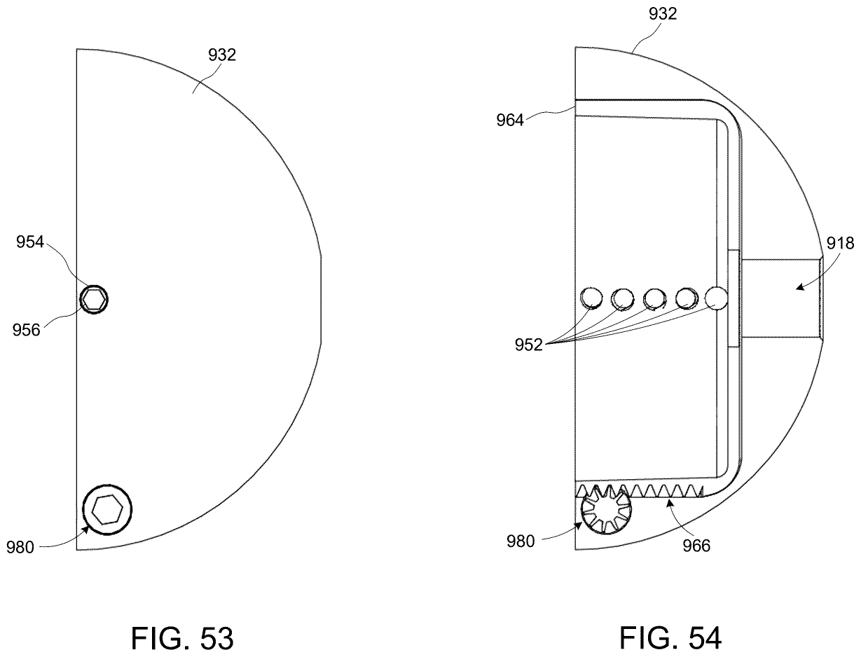

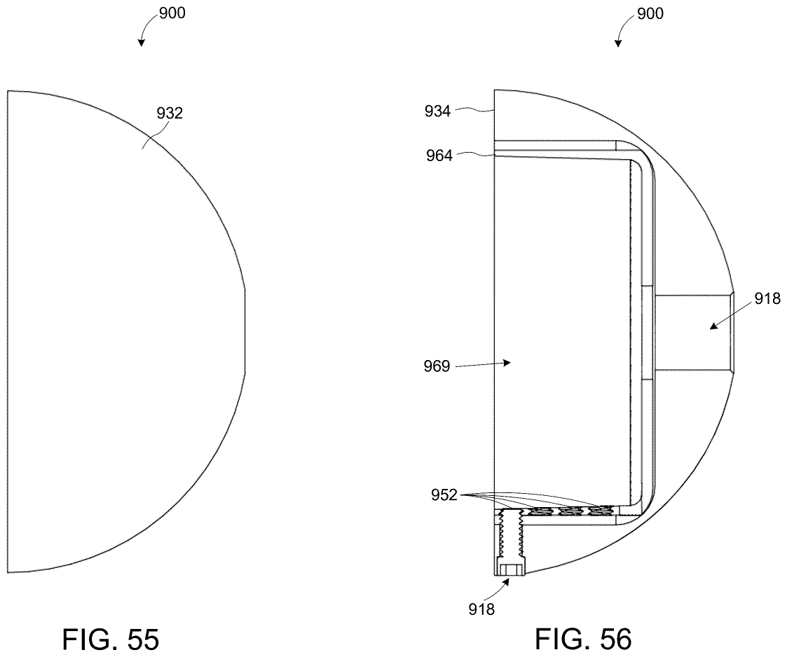

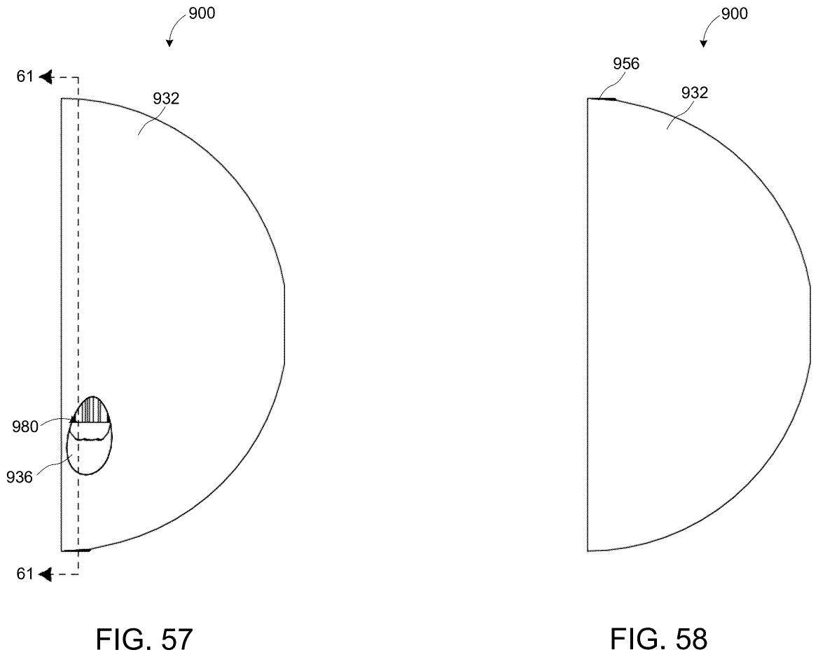

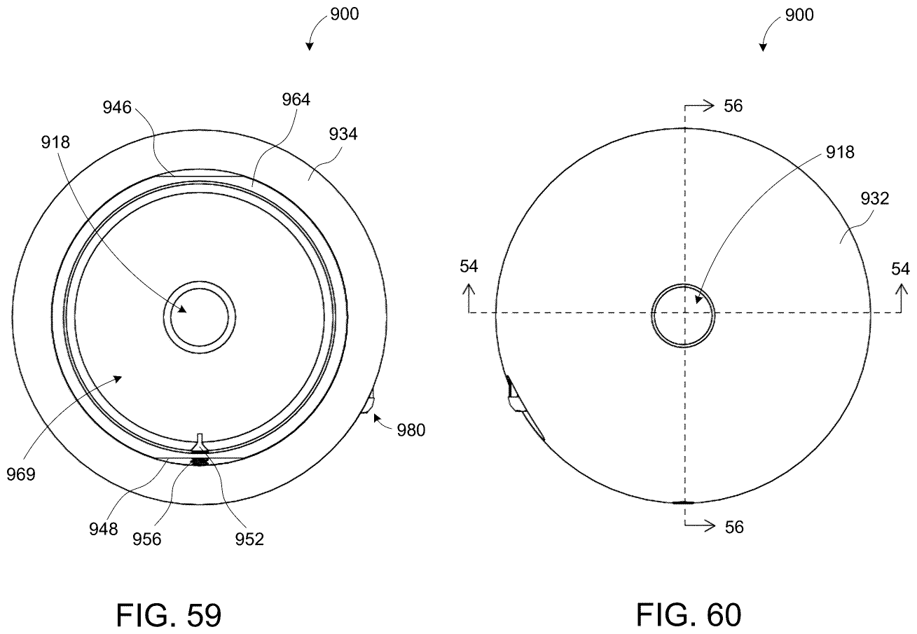

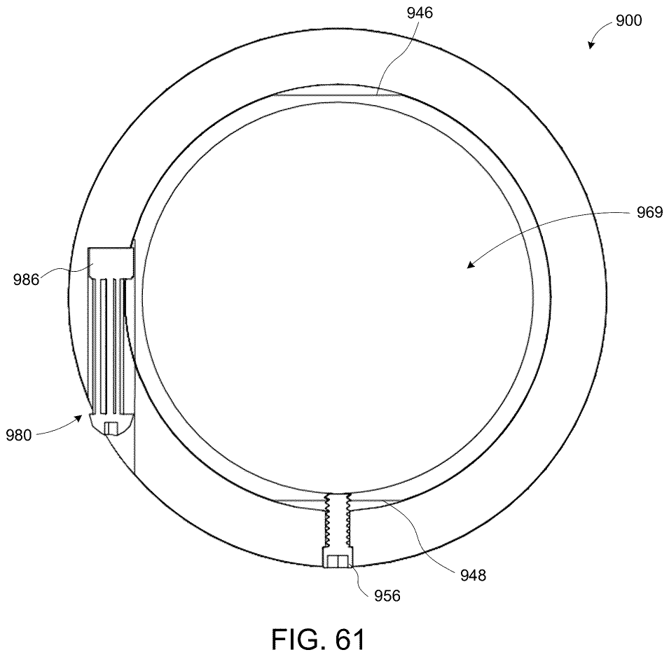

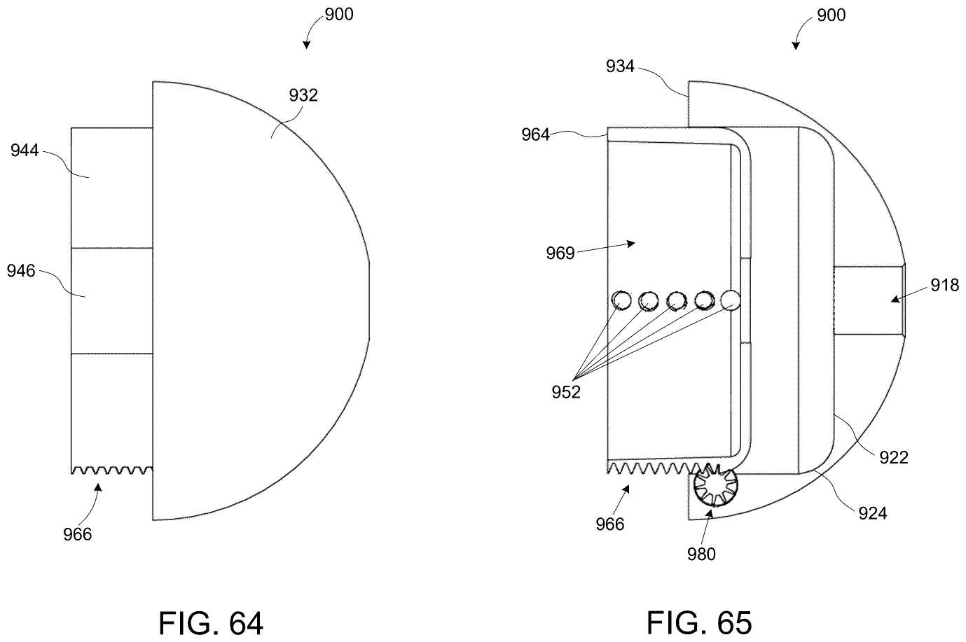

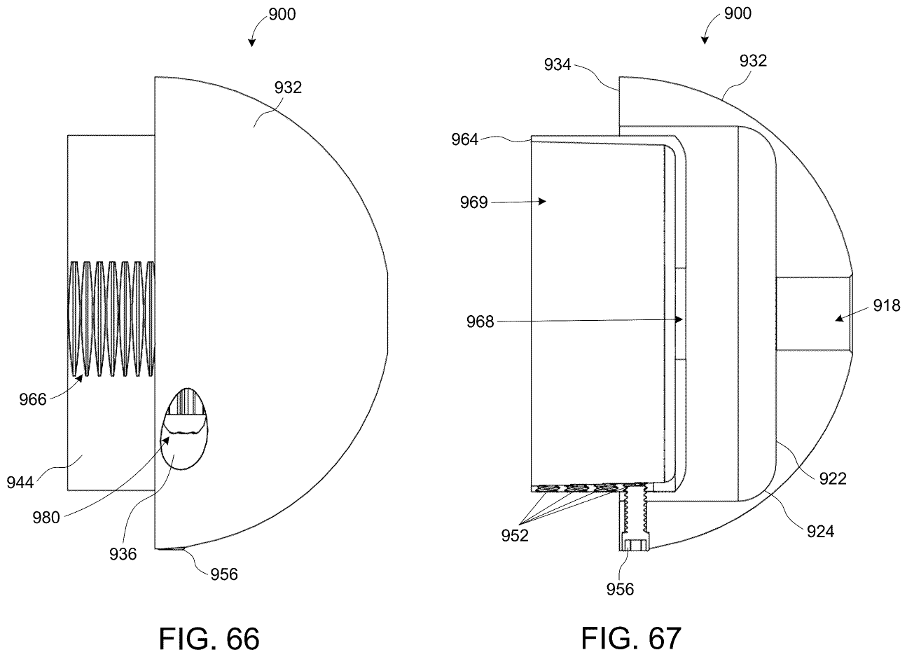

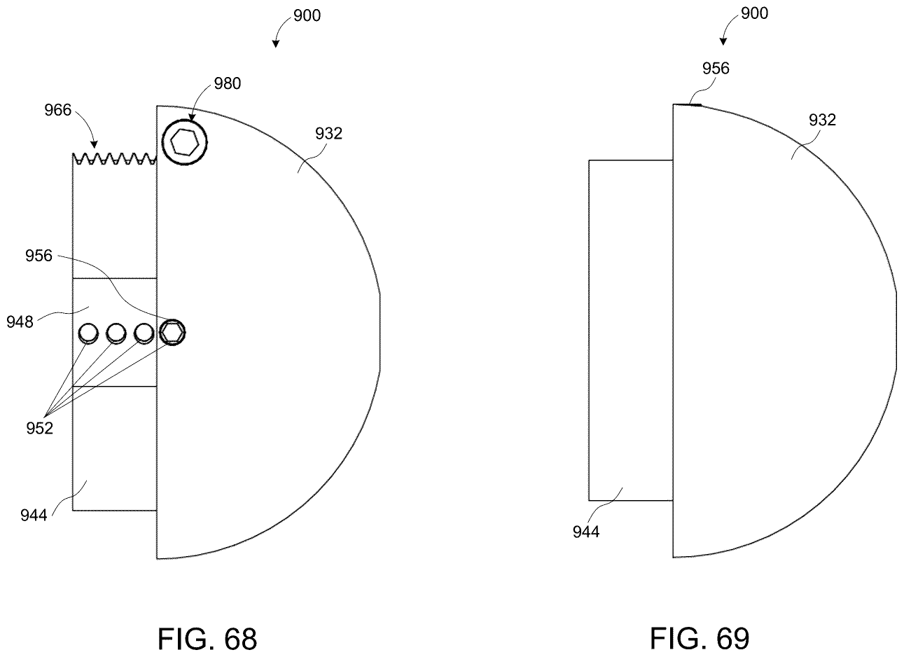

With particular reference to FIGS. 43-69, there is illustrated an alternative embodiment of an implant 900 for a glenoid implant system, which may be a traditional or reverse glenoid implant system. The implant 900 may have a head 930, a base member 960, and an extension/distraction system or movement system 950. The implant 900 may have a first end 902 at an end of the head 930 and a second end 904 at an opposite end of the base member 960.

With continued reference to FIGS. 43-50, the head 930 may have an articulating surface 932 and a base surface 934 opposite and extending between the articulating surface 932 on an interior surface of the head 930. The articulating surface 932 may have, for example, a hemispherical surface or shape. The articulating surface 932 may include a first opening 918 extending into the articulating surface from a first surface 902 towards the second end 904 of the implant 900. The base surface 934 may include, for example, a cavity 920 extending into the base surface 934 towards the first end 902 of the implant 900, defining a circumferential inner wall 926. The cavity 920 may extend a first distance towards the first end 902 of the implant 900, defining an inner top surface 922. The inner top surface 922 and the inner wall 926 may be connected by an inner filleted surface 924. The first opening 918 may extend into the head 930 a second distance to the surface 922. The first distance may be, for example, larger than the second distance. The first opening 918 and the cavity 920 may be, for example, cylindrical, where the first opening 918 may be concentrically aligned with the cavity 920. The first opening 918 may have a first diameter and the cavity 920 may have a second diameter. The first diameter of the first opening 918 may be smaller than the second diameter of the cavity 920. The articulating surface 932 may also include a second opening 954 extending a third distance approximately normally from the articulating surface 932 towards the inner wall 926. The second opening 954 may extend through the articulating surface 932 perpendicular to the first opening 918 and the cavity 920. The second opening 954 may be approximately circular. The articulating surface 932 may further include a second cavity 936 extending from the articulating surface 932 towards an opposite side of the articulating surface 932 at a fourth distance, intersecting the inner wall 926. The second cavity 936 may extend through the articulating surface 932 perpendicular to the first opening 918 and the cavity 920. The fourth distance of the second cavity 936 may be, for example, approximately the same as the third distance of the second opening 954. In addition, the first distance of the first opening 918 may be, for example, approximately the same as the fourth distance of the second cavity 936 and the third distance of the second opening 954. It is also contemplated that the first distance of the first opening 918 may be, for example, larger or smaller than the fourth distance of the second cavity 936 and the third distance of the second opening 954. In addition, the articulating surface 932 may include a third cavity 938 extending from an end of the second cavity 936 between the articulating surface 932 and the inner wall 926 towards the articulating surface 932 at a fifth distance. The third cavity 938 may have a fifth diameter smaller than the fourth diameter of the second cavity 936.

The base member 960 may have a first surface 962 at an end and a base or second surface 964 at an opposing end. The base surface 964 may be connected by a plurality of circumferential surfaces extending towards the first surface 962, i.e. a first circumferential surface 942, a second circumferential surface 944, a third circumferential surface 946, and a fourth circumferential surface 948. The first surface 962 may be connected to the plurality of circumferential surfaces 942, 944, 946, 948 by a filleted or angled surface 940. The first circumferential surface 942 may be opposite the second circumferential surface 944. The second circumferential surface 944 may have a plurality of fins 966 through a series of cuts into or protrusions out from the second circumferential surface 944. The plurality of fins 966 may extend, for example, parallel to the first surface 962 and the second surface 964. Each of the plurality of fins 966 may be approximately similarly sized and/or shaped and may be sequenced from an edge of second circumferential surface 944 to an opposing edge of the second circumferential surface 944. The plurality of fins 966 may contain, for example, 9 fins (or more, or less). The third circumferential surface 946 may be opposite the fourth circumferential surface 948. The third circumferential surface 946 and the fourth circumferential surface 948 may be, for example, approximately flat or concaved into the base member 960. The fourth circumferential surface 948 may have a plurality of through holes 952. The plurality of through holes 952 may be threaded. The plurality of through holes 952 may be aligned from an edge of the fourth circumferential surface 948 to an opposing edge of the fourth circumferential surface 948. There may be, for example, five through holes 952, although alternative numbers of through holes 952 are also contemplated based on the size of the base member 960 and/or desired increments of expansion of the implant 900. A cavity 969 may extend from the base surface 964 towards the first surface 968. The first surface 962 may include an opening 968. The cavity 969 may have a second diameter larger than a first diameter of the opening 969. The cavity 969 may also extend a second distance further than a first distance that the opening 968 extends between the first end 962 and the second end 964. The cavity 969 and the opening 968 may be, for example, approximately concentrically aligned.

The implant 900 may also include a screw 956 and a finned screw 970 for securing the implant 900 in a desired position or extension. The finned screw 970 may have a head 972, a plurality of fins 978, and a base member 980. The head 972 may have an opening with any type of screw driver opening, for example a hexagon (Allen), a torx, etc. The head 972 may be connected to the plurality of fins 978 at a first end. The plurality of fins 978 may extend radially outwards from a centerline of the screw towards an outer edge of the head 972 and an outer edge of the base member 980. The head 972 and the base member 980 being concentrically aligned. There may be any number of fins in the plurality of fins 978, for example approximately ten, although alternative numbers of fins 978 are also contemplated based on the length of the screw 970. The base member 980 may have a shaft 982 coupled to and extending from a body 986. The body 986 may be, for example, coupled to and extend from a second end of the plurality of fins 978. The body 986 may also have a first diameter and the shaft 982 may have a second diameter and the first diameter may be larger than the second diameter. The shaft 982 may be, for example, threaded or otherwise textured to engage the third cavity 938 of the head 930.

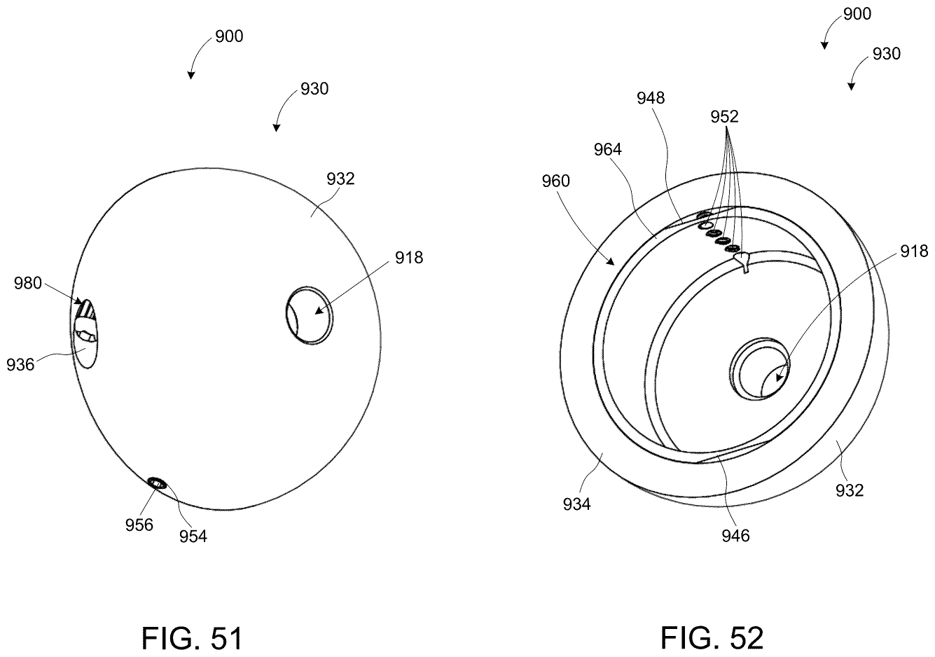

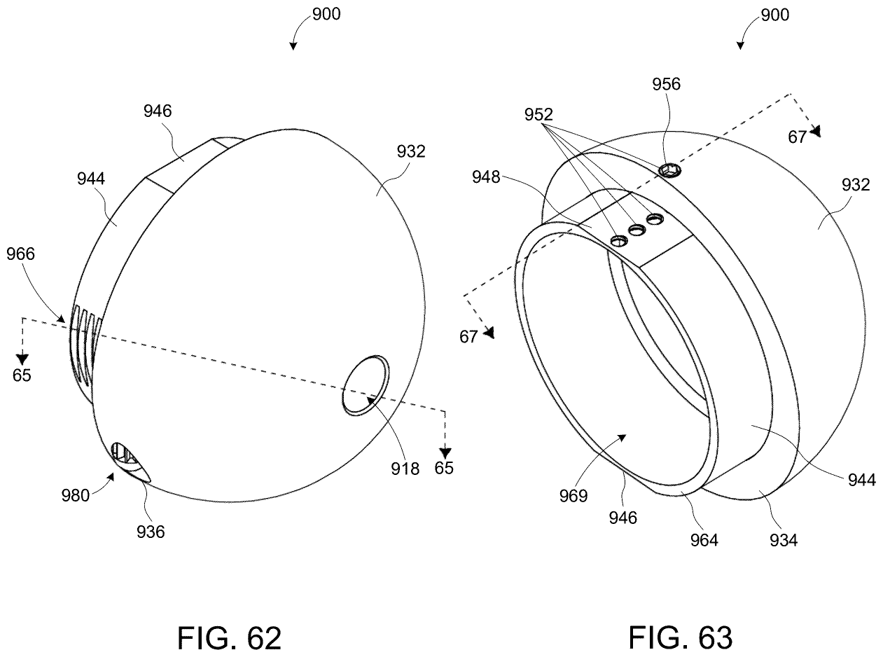

The implant 900 may further include an extension/distraction system 950. The extension/distraction system 950 may include the plurality of through holes 952, the finned screw 970, the cavities 936, 938 of the head 930, the second opening 954 of the head 930, the screw 956, and the plurality of fins 966. The extension/distraction system 950 may adjust a distance between the first end 902 of the implant 900 and the second end 904 of the implant 900. Referring to FIGS. 51-61, the distance between the first end 902 of the implant 900 and the second end 904 of the implant 900 may be at its shortest when the base member 960 is completely received within the cavity 920. The extension/distraction system 950 may operate by securing a relative position of the base member 960 to the head 930 by inserting the screw 956 through the second opening 954 and screwing the screw 956 into one of the plurality of through holes 952. As an example of the embodiment shown in FIGS. 51-61, the screw 956 may be inserted into one of the plurality of through holes 952 located nearest the first surface 962 and the filleted surface 940. Further, the extension/distraction system 950 may insert the finned screw 970 through the second cavity 936 and the shaft 982 of the finned screw 970 may be inserted into the third cavity 938. As illustrated in FIGS. 54 and 65, the fins 978 may align with the plurality of fins 966 of the base member 960, such that one or multiple of the fins 978 of the finned screw 970 may each be inserted in between two fins of the plurality of fins 966 of the base member 960. The opening in the head 972 of the finned screw 970 may be, for example, aligned with and accessible through the second cavity 936. A driver (not shown) may be inserted into the second cavity 936 and engage the opening in the head 972 of the finned screw 970. As the opening in the head 972 of the finned screw 970 is turned, the screw 970 rotates and the fins 978 engage the fins 966 of the base member 960 to move the base member 960 relative to the head 930. For example, rotating the finned screw 970 in a first direction could expand the implant 900, while rotating the finned screw 970 in a second direction could contract the implant 900. Referring to FIGS. 62-69, the distance between the first end 902 of the implant 900 and the second end 904 of the implant 900 may be at its longest length when the screw 956 is inserted into one of the plurality of through holes 952 located nearest the second end 904 of the implant 900. A user may dynamically adjust a length between the first end 902 and the second end 904 of the implant 900 by using the finned screw 970 to expand or contract the implant 900. Once the desired expansion of the implant 900 is achieved, a screw 956 may be inserted into one of the pluralities of through holes 952 to lock the implant 900 in the desired position.

In an example of a use of inserting an implant 100, 300, 500, 900 using a standard deltopectoral approach, a patient may be placed in a desired position for surgery. Alternatively, superior or subscapularis approaches may be used. Next, any preoperative markings or planning may be performed. A surgeon may then make an incision and in a deltopectoral approach the surgeon may move any soft tissue to expose the patient's joint. For example, the deltoid may be retracted laterally and the pectoralis and conjoined tendon may be retracted medially. Once the glenohumeral joint has been accessed and stabilized, the surgeon may then remove the humeral head by making a cut at the desired position with or without a cut guide. Next, the surgeon may then expose the glenoid by retracting the proximal humerus posteriorly. Additional preparation may also be performed, for example, excising residual labral tissue, releasing the biceps tendon, and releasing the capsule from the glenoid anteriorly, inferiorly, and posteriorly. Appropriate glenoid retractors may then be inserted and additional exposure techniques can then be used, as needed.

Then, the glenoid surface may be resected to create a flat base. The surgeon may prepare the glenoid surface with instruments, such as drill(s), reamers and the like, to prepare the glenoid to receive a base member 160, 360, 560, 960. For example, pin guides may be used by referencing the face of the glenoid and appropriately seating the assembled pin guide on the inferior edge of the glenoid to reduce the risk of impingement. A guide may then be drilled through the pin guide handle until bi-cortical fixation is achieved. Next, the flat glenoid surface may be formed using a cannulated baseplate reamer of the same diameter of the baseplate that will be used with the patient. Either a full-moon or a half-moon reamer may be used to obtain complete seating and secure fixation of the glenoid baseplate. Once a reamer is chosen, the reamer may be slide onto the guide pin and the glenoid surface may be reamed. A hole for the baseplate post may then be drilled over the guide pin using a cannulated drill bit. After the hole is drilled, the guide pin may be removed and the surgeon may determine the length of the post and/or diameter of the central screw needed for the base member 160, 360, 560, 960. In addition, the surgeon will select the base member 160, 360, 560, 960 based on the diameter of the reamed glenoid surface.

The base member 160, 360, 560, 960 may be inserted onto the glenoid surface. The base member 160, 360, 560, 960 may be inserted by screwing or impacting the shaft 166, 366, 566 or other central screw into the prepared hole in the glenoid until the base member 160, 360, 560, 960 is fully seated against the surface. Then, the surgeon may choose to secure the base member 160, 360, 560, 960 to the glenoid surface through various means such as screws, hammers, and the like. Then, if the base member 160, 360, 560, 960 is not already connected to or needs to be coupled to the head 130, 330, 530, 930, respectively, the surgeon may then attach the head 130, 330, 530, 930 to the base member 160, 360, 560, 960. To adjust a distance between the first end 102, 302, 502, 902 and the second end 104, 304, 504, 904, the surgeon may extend or distract the extension/distraction mechanism and/or system 150, 350, 550, 950. The implants 100, 300, 500 may be extended or distracts using, for example, the surgeon's hands or a tool, such as, a distractor implement 700. Alternatively, for the implant 900, the surgeon may extend or distract the implant 900 by using a driver (not shown) engaged with the head 972 of the finned screw 970. Once a desired length of the implant 100, 300, 500, 900 has been achieved, the surgeon may choose to insert one or more spacers 140 in between the base surface 134, 334, 534 of the head and the first surface 162, 362, 562 of the base member. The surgeon may couple the one or more spacers 140 to other spacers and/or the base surface 134, 334, 534 of the head and the first surface 162, 362, 562 of the base member, as described above. The surgeon may then compress the base member 160, 360, 560 to the head 130, 330, 530. If the humerus does not need to be replaced, then the surgeon can proceed with completing the surgery. However, if the humerus also needs to be replaced, then, the surgeon may prepare the humerus to provide a corresponding articulating surface for engaging the articulating surface 132, 332, 532, 932 of the glenoid implants 100, 300, 500, 900, as known by one of ordinary skill in the art. For example, a peripheral reamer associated with the corresponding diameter of the chosen glenosphere may be attached to a T-handle. The reaming may be performed manually and kept parallel to the central screw. The pilot tip on the reamer may be carefully inserted into the central hole of the base member 160, 360, 560, 960 in alignment with the axis of the shaft 166, 366, 566 or central screw of implant 900. Then, manual reaming is performed using a back and forth sweeping motion.

After the glenoid implant 100, 300, 500, 900 is inserted into the glenoid and the humerus is ready to receive the glenoid implant 100, 300, 500, 900, then the surgeon may position the head 130, 330, 530, 930 of the implant 100, 300, 500, 900 onto the surface of the humerus. In some embodiments, it is also contemplated that, for example, a poly or plastic spacer may be positioned between the glenoid implant and the humerus implant to create a relatively smooth articulating joint. Next a surgeon may test the connection between the base member 160, 360, 560, 960 and the head 130, 330, 530, 930. If the surgeon decides that the connection between the head 130, 330, 530, 930 and the base member 160, 360, 560, 960 needs to be adjusted, the surgeon may distract the extension/distraction mechanism 150, 350, 550 to the new desired length and re-insert or remove the one or more spacers 140, as needed. Alternatively, for the implant 900, the surgeon could use a driver to turn the screw 970 and expand or retract the implant 900 to the new desired length. Once the desired length and connection has been made for the implant 100, 300, 500, 900 and the shoulder joint is in the desired position, the surgeon may then repair or reposition any soft tissue surrounding the joint. Once the lateralization and distalization of the joint are complete, then the tension and stability of the implant 100, 300, 500, 900 may be tested. After the joint and all surrounding tissue has been repositioned and the testing shows the desired tension and stability, the surgeon may close the incision. Though these steps have been explained in a specific order, the surgeon may change, omit, or add steps as the surgeon sees fit, as various steps may arise due to a plurality of factors such as complications during the surgery, different anatomical specifics of each patient, and the like. Further, if complications arise post-surgery, the implant 100, 300, 500, 900 may be re-tensioned or adjusted by repeating the steps above to open up the patient's body and adjust a length of the implant and possibly re-insert one or more spacers 140 as appropriate.

The implant 100, 300, 500, 900 including the head 130, 330, 530, 930 and the base member 160, 360, 560, 960 may be made from various materials. For example, metal parts of the implants, such as the base member 160, 360, 560, 960 may be made from stainless steel, titanium alloys, and/or cobalt-chromium alloys. Plastic components of the implants may be made from medical-grade polyethylene, such as the articulating surface of the humerus. Other components of the implant may use ceramics or ceramic/metal mixtures, such as oxidized zirconium. A choice of material may also be dependent on the specific patient's needs.

Although the invention described above has been described in its use for a glenohumeral joint, the principles of a dynamically adjustable implant for a ball and socket joint that this disclosure teaches may be used in other parts of the body, such as the hip and/or coxofemoral joint.

As may be recognized by those of ordinary skill in the art based on the teachings herein, numerous changes and modifications may be made to the above-described and other embodiments of the present disclosure without departing from the scope of the disclosure. The components of the implants, devices, and/or systems as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the implants, devices, and/or systems may include more or fewer components or features than the embodiments as described and illustrated herein. For example, the components and features of implants 100, 300, 500, 900 may be used interchangeably and in alternative combinations as would be modified or altered by one of skill in the art. The distractor 700 or a device using the same principles taught within this application may be used for any embodiment of a glenoid implant/reverse glenoid implant 100, 300, 500 described herein. Further, the steps of the surgical methods associated with the implants 100, 300, 500, 900 may be used interchangeably and in alternative combinations as would be modified or altered by one of skill in the art. Accordingly, this detailed description of the currently preferred embodiments is to be taken illustratively, as opposed to limiting the disclosure.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

Having thus described the preferred embodiments, the invention is now claimed to be:

1. An implant, comprising:
a head;
a base member moveably coupled to the head; and
a movement system positioned within a portion of the head and engaging a portion of the base member.

2. The implant of claim 1, wherein the head comprises:
an articulating surface extending from a first end of the head to a second end of the head on an exterior surface; and
a base surface on the second end of the head.

3. The implant of claim 2, wherein the base surface comprises:
a first cavity inset into the base surface of the implant opposite the articulating surface forming an inner wall of the head.

4. The implant of claim 3, wherein the head further comprises:
a second cavity extending into the inner wall of the head towards the articulating surface;
a third cavity extending from an interior end of the second cavity into the head;
wherein the second cavity and the third cavity receive a portion of the movement system.

5. The implant of claim 4, wherein the head further comprises:
a fastener opening extending through the head from the articulating surface to the inner wall; and
an opening extending through the head from the articulating surface to the first cavity near a central portion of the head.

6. The implant of claim 5, wherein the base member comprises:
a first surface at a first end;
a base surface at a second end opposite the first end; and
a plurality of circumferential surfaces extending between the first surface and the base surface.

7. The implant of claim 6, wherein the base member further comprises:
an angled surface extending between the first surface and the plurality of circumferential surfaces.

8. The implant of claim 6, wherein the plurality of circumferential surfaces of the base member further comprises:
a first circumferential surface;
a second circumferential surface opposite the first circumferential surface;
a third circumferential surface positioned between a first end of the first circumferential surface and a first end of the second circumferential surface; and
a fourth circumferential surface positioned between a second end of the first circumferential surface and a second end of the second circumferential surface, wherein the third circumferential surface is positioned opposite the fourth circumferential surface.

9. The implant of claim 8, wherein the second circumferential surface comprises:
a plurality fins extending from a portion of an exterior surface of the second circumferential surface, and wherein the plurality of fins extend parallel to the first surface of the base member.

10. The implant of claim 9, wherein the first circumferential surface and the second circumferential surface are curved and wherein the third circumferential surface and the fourth circumferential surface are flat.

11. The implant of claim 9, wherein the fourth circumferential surface comprises:
a plurality of through holes positioned in a row between the first surface and the base surface of the base member.

12. The implant of claim 11, wherein the base member further comprises:
a cavity extending into the base member from the base surface.

13. The implant of claim 11, wherein the plurality of through holes of the fourth circumferential surface of the base member align with the fastener opening of the head.

14. The implant of claim 9, wherein the movement system comprises:
a finned screw positioned within the second cavity of the head.

15. The implant of claim 14, wherein the finned screw further comprises:
a head at a first end;
a base member at a second end; and
a plurality of fins extending radially outward between the head and the base member.

16. The implant of claim 15, wherein the finned screw further comprises:
a shaft extending away from the base member on a side opposite the plurality of fins, and wherein the shaft has a first diameter and the base member has a second diameter and wherein the first diameter is smaller than the second diameter.

17. The implant of claim 16, wherein the shaft is positioned within the third cavity of the head and the head, the base member, and the plurality of fins is positioned within the second cavity of the head.

18. The implant of claim 17, wherein the plurality of the fins of the finned screw rotatably engage the plurality of fins of the base member, and wherein the finned screw further comprises:
a drive opening extending into the head at the first end of the finned screw.

19. The implant of claim 13, further comprising:
a fastener inserted through the fastener opening of the head and into at least one of the plurality of through holes of the base member.

20. A method of adjusting an implant in a joint, comprising:
using an extension/distraction mechanism to position the implant at a desired length comprises:
inserting a driver into a finned screw of the implant to engage the finned screw; and
rotating the driver and the finned screw to adjust a head of the implant relative to a base member of the implant at a desired length.
